(19) United States
(12) Patent Application Publication
(10) Pub. No.: US 2023/0005608 A1
(43) Pub. Date: Jan. 5, 2023

(54) MANAGEMENT AND TRACKING OF PATIENT ENCOUNTERS AND MEDICAL INFORMATION INCLUDING IMMUTABLE TOKENS, DIGITAL ASSETS AND SMART CONTRACTS

(71) Applicant: Scientia Potentia Est II, LLC, Charleston, SC (US)

(72) Inventors: Jeremy Blackburn, Charleston, SC (US); Justin Southward, Fort Meyers, FL (US); W. Kurt Taylor, N. Charleston, SC (US); Karl David, Charleston, SC (US); Austi Critchfield, Clearwater, FL (US); Michael Lu, N. Charleston, SC (US); Tim McVicker, Charleston, SC (US); Bryan McArdle, Charleston, SC (US)

(73) Assignee: Scientia Potentia Est II, LLC, Charleston, SC (US)

(21) Appl. No.: 17/900,624

(22) Filed: Aug. 31, 2022

Related U.S. Application Data
(63) Continuation-in-part of application No. 17/866,256, filed on Jan. 1, 0001.
Application No. 17/866,256 is a continuation-in-part of application No. 17/697,839, filed on Mar. 17, 2022.
Application No. 17/697,839 is a continuation-in-part of application No. 17/686,502, filed on Mar. 4, 2022.
Application No. 17/686,502 is a continuation-in-part of application No. 17/566,957, filed on Dec. 31, 2021.
Application No. 17/566,957 is a continuation-in-part of application No. 17/561,827, filed on Dec. 24, 2021.
Application No. 17/561,827 is a continuation-in-part of application No. 17/531,746, filed on Nov. 20, 2021, now Pat. No. 11,482,325.
Application No. 17/531,746 is a continuation-in-part of application No. 17/531,598, filed on Nov. 19, 2021.
Application No. 17/531,598 is a continuation-in-part of application No. 17/344,043, filed on Jun. 10, 2021.
Application No. 17/344,043 is a continuation-in-part of application No. 17/230,911, filed on Apr. 14, 2021, now Pat. No. 11,288,761.
Application No. 17/230,911 is a continuation-in-part of application No. 17/176,056, filed on Feb. 15, 2021, now Pat. No. 11,288,308.
Application No. 17/176,056 is a continuation-in-part of application No. 17/128,084, filed on Dec. 19, 2020.
Application No. 17/128,084 is a continuation-in-part of application No. 16/997,840, filed on Aug. 19, 2020, now Pat. No. 11,449,949.
Application No. 16/997,840 is a continuation-in-part of application No. 16/994,585, filed on Aug. 15, 2020, now Pat. No. 11,232,652.
Application No. 16/994,585 is a continuation-in-part of application No. 16/991,916, filed on Aug. 12, 2020, now Pat. No. 11,216,823.
Application No. 16/991,916 is a continuation-in-part of application No. 16/876,080, filed on May 17, 2020, now Pat. No. 11,423,360.
Application No. 16/876,080 is a continuation-in-part of application No. 16/810,782, filed on Mar. 5, 2020, now Pat. No. 11,216,781.
Application No. 16/810,782 is a continuation-in-part of application No. 16/510,634, filed on Jul. 12, 2019, now Pat. No. 10,713,737.
Application No. 16/510,634 is a continuation-in-part of application No. 16/510,542, filed on Jul. 12, 2019, now abandoned.
Application No. 16/510,634 is a continuation-in-part of application No. 16/452,076, filed on Jun. 25, 2019, now abandoned.

(51) Int. Cl. G16H 40/20; G16H 20/10; G16H 40/67; G16H 15/00; G16H 10/60; G06Q 30/00

(52) U.S. Cl. CPC G16H 40/20; G16H 20/10; G16H 40/67; G16H 15/00; G16H 10/60; G06Q 30/0185; G06Q 2220/00

(57) ABSTRACT

This system is directed to a computerized system having a computer system; an identification device; a medical monitor; an immutable storage system and a set of non-transitory portable computer readable instructions included in the computer system adapted for: receiving a medical provider information, representing a medical provider from the identification device wherein the medical provider information includes a medical provider information date, a medical provider information time and a medical provider information location of a medical information capture, receiving a medical information from the medical monitor wherein the medical information includes a patient information, a medical information date, a medical information time and a medical information location of a medical capture, creating a current medical encounter record according to the medical provider information and medical information, and, storing the current medical encounter record in the immutable storage system.

### BACKGROUND

### 1) Field of the System

A computerized system for management of patient encounters including pairing physical activities, events, objects, and individuals and, associated virtual representation to verify a link between the activity and object with a virtual representation to create an undichotomized pair that can be used for authentication, verification, anti-fraud, appraisals, auditing, recording—and the like—of the status, activity, and transactions associated with the medical device, task, medication, treatment, or other medical activity. A system for pairing medical events and objects utilizing location data, timing of when they occur, biometric identity of the patient or participant, biometric identification of the provider or medical personnel including service and activity identification. The system pairs an occurring event with a digital representation that can be adapted for use with health care, medical records, insurance, billing, government oversight, verification, or other administrative process. The verified digital data collected can be used to effectuate payments, reimbursements, and smart contracts to facilitate automatic transactions between entities as well as to automatically detect, determine, and record compliance with requirements, private and governmental. The verified digital data can also be used to create digital assets, non-fungible tokens and provide information to third parties.

### 2) Background

The medical industry is a large and complicated industry for tracking data, activities, individuals, and objects. For example, there is a significant market for medical devices and medications including controlled, restricted, and prescribed medications. Further, some medical activities have increased risk, value, need for control, are prone to mistakes, fraud, and can be subject to abuse.

When performing medical activities, the medical provider goes through a process of reimbursements, which is a process used for medical provider to receive payments for providing healthcare or medical services. To reduce the time between the medical activity and the payments, the process can start before the medical activity is administered or provided and can continue until after its completion. A system that allowed for data tracking prior to a medical activity that can associate the tracked data with medical activity would be advantageous.

It has been reported that fraudulent prescription drugs are subject to more abuse in older adults and, this has been a growing problem. It would be advantageous to have a system that better controlled the provisioning of such drugs and, from the medical provider to the patient, to reduce or eliminate prescription drug abuse and reduce or eliminate reimbursement for medications that are not actually provided to the patient. While there have been efforts to address this problem, these efforts are subject to many exposing issues that result in mistakes in the making, distribution, prescription, dispensing, and usage of drugs. According to the National Center for Biotechnology Information, each year in the United States alone, 7,000 to 9,000 people die because of medication errors. Further, it is estimated that the abuse of opioid analgesics alone results in over $72 billion in medical costs each year. There is also a rise in healthcare fraud, with one entity having to pay $270 million to resolve a fraudulent claim. One estimate of Medicaid fraud states that the waste and loss accrued exceeding $25 billion annually.

The issues with verification that medical services were provided, proper medications were prescribed and delivered, the proper medical personnel administered or provided the medical services, are some issues that if not properly managed, can lead to mistakes, fraud, and abuse. The problem is compounded with the lack of effective regulation and weak enforcement capacity. Further, as the industry becomes more digital, the opportunity for undesirable events occurs such as data breaches and identity theft that can be used to improperly receive medications. A system that is based up on verifiable truths rather than trust would be advantageous.

In application, Medicare, Medicaid, and other insurance companies are susceptible to fraud which results in unnecessary costs to those companies as well as taxpayers, which can be in the billions of dollars per year. A system that creates an immutable record that verifies the medical provider or professional, patient, medications, location, activity, treatment, event, object, and other items, and functions to pair these with a digital representation that does not rely exclusively upon trusting the digital representation or inputted data alone would be advantageous. The digital representation can represent medical devices, medications, treatments, locations, providers, healthcare workers, patients, participants, and the like, so that an immutable record is provided that can reduce, if not eliminate, fraud. Verifying who, what, when, and where, as it relates to a medical object, and storing it in an immutable database can provide for truth in data, autonomous processing of not only billing and payments but other administrative processes and provide a more secure data storage system. Autonomous verification and storage of digital representations would not only save money and time related to unnecessary costs and fraud, but also would expedite payments resulting in better cash flows and ease of auditing.

There is also an issue with a chain of title or custody for articles such as medical devices, medicines, and medical containers, and the association of these to individuals. Current systems have a lack of accountability, verification, and reliability of information related to the article and transactions. The inability to verify or authenticate articles and other factors can result in loss, mistakes, increased insurance claims, fraud, increased insurance premiums, injury, and death. While there have been some attempts to add item information to a physical material, such as U.S. Pat. No. 8,321,302, these attempts have focused on tracking inventory levels and do not include verifiably pairing a physical material with a virtual representation that can be tracked throughout a process. Further, these prior attempts focus on a single location and do not consider the fact that the physical location of the article can be at separate locations, from creation to distribution and use. This disadvantage can be seen in U.S. Pat. No. 8,521,620 which specifically states that if a RFID tag is lost or damaged, the system allows a user to enter an item number or style and tags of similar items are displayed, a new tag is generated and associated with the item having the lost or damaged tag. Once the RFID tag is lost or damaged, the medications or container is no longer paired. While this system specifically allows for the replacement of RFID tags on the same item, it lacks the ability to verifiably pair the new tag with the medications or container. A system immutably pairing individuals, services, activities, objects, and any combination would be advantageous.

The disadvantages of current systems are caused in part due to the lack of an immutable storage system associating the medical device, medication, or container with the virtual representation to determine that the article promised and delivered are properly paired, both physically and digitally. Attempts to provide for inspections (e.g., authentications) that an article is original has not resolved the existing set of problems. There is also a need to verify that the individuals, during the creation, authentication and transactions, are who they say that they are, and identify what tasks they have completed. Evidence that the existing systems fail to address the current and obvious problem is that in 2021, the United States federal government won or negotiated more than $5 billion in healthcare fraud judgments and settlements, the largest amount ever in the history of Health and Human Services and Department of Justice's fraud and abuse enforcement program.

One such system that has failed to provide a meaningful solution is found in United States Patent Application 2014/0288958 which discloses systems and methods that are described to facilitate providing an online automated payment architecture that aggregates funds from multiple health-related accounts (e.g., a member's HSA, an employer's advance account, etc.) and automatically transfers the aggregated funds to a healthcare provider's account to pay a bill or invoice. This system fails to provide a verifiable link to the actual individuals, activities, and actions with the digital representations. Further, this system does not track the activity, individual, date, time, and location to reduce or prevent fraud, nor does it provide efficiencies in autonomous processes such as smart contracts.

There is also a need to verify that the actions being performed by the individual are in compliance with applicable standards, regulations and other requirements. In some reimbursement systems, reimbursement is only provided if the medical provider meets the requisite authorization, licensees, and other criteria. For example, when a skilled nursing facility provides services covered by Medicare (e.g., speech therapy, physical therapy, occupational therapy), Medicare skilled nursing facility reimbursement rates are calculated under the prospective payment system through Medicare part A so that verification of the medical providers' status and licenses are important to payments. A system that can provide a warning when the criteria are not met, at the time of provision of medical services, would be advantageous. Specifications in some processes can include the specification of an authentication process, commercialization process, or other performance criteria of the article. Specifications can also include initial registration and transaction requirements, as well as notifications.

It would be advantageous to have a system that could have medications and/or medical device specifications or requirements related to such things as patient vitals, time of administration of dosage, or licensure and/or training of the individuals involved in the event in an immutable data record where a system could compare the conditions at the time of the event to the specifications and requirements of the medication or medical device. An immutable record of the event would be useful for future diagnoses and medical evaluations of the patient or persons involved in the event as well as tracking for insurance or other purposes.

It would be advantageous to have a system that verified articles, authorized individuals, verified proper environmental parameters as well as verified that items and tasks are properly paired with a virtual representation. It would be advantageous to have a system that reduced or eliminates the risk of a counterfeit, fraudulent, substitute, lower quality, or other non-designated or approved article being used. It would be advantageous for a system that prevented or reduced the risk of counterfeit, unlicensed or unauthorized articles.

It would be advantageous to have a system that allows for third party or automated independent verification to reduce counterfeit, fraudulent, false, or misleading information and activity.

It would be an advantage to have a system that can pair medical activities with virtual representation so that authentication can be easily and quickly accomplished in real time.

It would be advantageous to have a system that provides for multi-party verification for tracking of the medical activities, individuals, and physical articles. This tracking could happen autonomously and with location confirmation.

It would be advantageous to have a system that provides for a verified trust association between medical activities, articles, and virtual representations.

It would be advantageous to have an immutable storage of a digital record of medical events that are paired with the patient or recipient, the provider or healthcare worker, as well as the facility, medicines, medical objects, and location whereby the event took place. This immutable record could then be accessed by additional stakeholders in a secure manner such as using a biometric key or unique security token. The token or key could be given to future medical providers, insurance or other administrative processes, or be accessed by the patient or other stakeholder themselves for future reference upon authorization.

The medical, pharmaceutical, and drug manufacturing industries are one of the most regulated business segments. Misrepresentation, fraud, and errors can lead to injury and even unnecessary death. A system that has improved verification and does not rely upon “trust” of the human operator or human input generated data would be desirable. The insurance industries are also rocked with fraud bloated management and oversight costs in multiple entries related to claims and services provided that have been billed. A platform that requires only a matching biometrically and that can automate autonomously the entire payment and confirmation process by verifications of when where and what and by whom would again be of great value.

It would be advantageous to have an immutable storage system whereby an auditing engine could confirm the data records being created. The data records, being immutable, would be verified as to the accuracy and confirmation of hashes, or other means immutably recorded, in the data as well as the data contained in the hashes being accurate. The data obtained could then be used for predictive analytics as well as for analytical processes to help improve medical treatments, patient experiences, productivity, efficiencies, optimizations, medicine, and product life cycles.

### SUMMARY OF THE SYSTEM

In accordance with an exemplary embodiment, this system can be a computerized system for management of patient encounters comprising: a medical encounter computer system in communication with an immutable storage system; a sensor in communication with the medical encounter computer system; a medical device in communication with the medical encounter computer system adapted to record vital signs of a patient; a healthcare provider record stored on the immutable storage system; a patient record stored on the immutable storage system; a set of computer readable instructions included in the medical encounter computer system adapted to: receive healthcare provider identifying information from the sensor, retrieve the healthcare provider record from the immutable storage system and generate a healthcare provider notification according to a comparison of the healthcare provider identifying information with the healthcare provider record, receive patient identifying information from the sensor, retrieve the patient record from the immutable storage system and generate a patient notification according to a comparison of the patient identifying information with the patient record, receive healthcare activity information from the sensor, and generate a healthcare activity notification according to a comparison of the healthcare activity information with the healthcare provider record, and, generate a medical device notification according to a comparison of a medical device usage with the healthcare provider record.

The system can include a sensor that is adapted to receive biometric information. The healthcare provider identifying information can include a healthcare provider location and, the set of computer readable instructions are adapted to receive a medical device location from the medical device and generate a medical device location notification according to a comparison of the healthcare provider location with the medical device location. The patient identifying information can include a patient location and the set of computer readable instructions are adapted to receive a medical device location from the medical device and generate a medical device location notification according to a comparison of the patient location with the medical device location. The patient identifying information can include a patient location, the medical encounter computer system includes a medical encounter computer system location, and the set of computer readable instructions are adapted to generate a medical device location notification according to a comparison of the patient location and the medical encounter computer system location. The medical encounter computer system includes a medical encounter computer system location, and the set of computer readable instructions can be adapted to generate a medical device location notification according to a comparison of a healthcare provider location received from the medical encounter computer system and the medical encounter computer system location. The medical encounter computer system can include a medical encounter computer system location, and the set of computer readable instructions are adapted to generate a medical device location notification according to a comparison of a patient location included in the patient record and the medical encounter computer system location.

The computerized system can include a patient record stored on the immutable storage system and a set of computer readable instructions included in the medical encounter computer system adapted to: receive healthcare provider identifying information from the sensor, retrieve the healthcare provider record from the immutable storage system, and generate a healthcare provider notification according to a comparison of the healthcare provider identifying information and the healthcare provider record. It can also receive patient identifying information from the sensor, retrieve the patient record from the immutable storage system, and generate a patient notification according to a comparison of the patient identifying information and the patient record, and receive a healthcare activity information from the sensor and generate a healthcare activity notification according to a comparison of the healthcare activity information and the healthcare provider record. The healthcare provider record can include healthcare provider authorization, and the set of computer readable instructions are adapted for generating an authorization notification according to a comparison of the healthcare provider record and the healthcare activity. The set of computer readable instructions can be adapted to receive a treatment location from the sensor, receive a healthcare provider location from the sensor, receive a patient location from the sensor and generate a location notification according to a comparison of the healthcare provider location, patient location information and healthcare activity location. The set of computer readable instructions can be adapted to receive a healthcare provider arrival time, receive a healthcare provider departure time, calculate an on-location time, and generate a temporal notification according to a comparison of the on-location time and the healthcare activity information. The set of computer readable instructions can be adapted to append the on-location time with the patient record to create an appended patient record and store the appended patient record on the immutable storage system. The healthcare provider arrival time can be a first healthcare provider arrival time and the set of computer readable instructions are adapted to: receive a first location associated with the healthcare provider departure time, receive a second healthcare provider arrival time at a second location, calculate a travel time according to the first location and the second location, and generate a travel notification according to a comparison of the healthcare provider departure time, the second healthcare provider arrival time, and the travel time. The set of computer readable instructions can be adapted to receive a healthcare provider arrival time, receive a healthcare provider current location information, retrieve a healthcare provider prior location information from the immutable storage system, calculate a travel time according to the healthcare provider arrival time and method of travel as well the healthcare provider prior location information, and generate a travel notification according to a comparison of the healthcare provider's arrival time and the travel time. The healthcare provider prior location information can be a prior activity information taken from a group consisting of parking information, transaction information, travel information, event information, mobile device location, mode of travel, and any combination thereof. The set of computer readable instructions can be adapted to create a medical encounter record according to the healthcare provider identifying information, the patient identifying information and the healthcare activity information. The sensor can be adapted to retrieve a medical device information from a medical device associated with the patient. The set of computer readable instructions can be adapted to retrieve a medication record from a medication computer system and generate a medication notification according to a comparison of the medication record and the patient record.

The computerized system can include a medication record stored on the immutable storage system and a set of computer readable instructions included in the medical encounter computer system adapted to: receive healthcare provider identifying information from the sensor, retrieve the healthcare provider record from the immutable storage system, and generate a healthcare provider notification according to a comparison of the healthcare provider identifying information and the healthcare provider record, retrieve the medication record and generate a medication notification according to a comparison of a patient record with the medication record and, receive a healthcare activity information from the sensor, retrieve a medical regulatory record from a regulatory computer system and generate a regulatory notification according to a comparison of the healthcare activity information and the medical regulatory record. In one embodiment, the system can use the locations of the patient, healthcare provider or other individual for determining milage which can include providing reimbursement functionality through smart contracts.

The set of computer readable instructions can be adapted to retrieve a healthcare activity time from the medical encounter computer system and generate a medication notification according to a comparison of the healthcare activity time with the medication record. The set of computer readable instructions can be adapted to retrieve a patient record from the immutable storage system and generate a medication notification according to a comparison of the patient record and the medication record. The set of computer readable instructions included in the medical encounter computer system adapted to: receive healthcare provider identifying information from the sensor, retrieve the healthcare provider record from the immutable storage system and generate a healthcare provider notification according to a comparison of the healthcare provider identifying information and the healthcare provider record, and, receive a healthcare activity information from the sensor, retrieve a medical regulatory record from a regulatory computer system and generate a regulatory notification according to a comparison of the healthcare activity information with the medical regulatory record.

The set of computer readable instructions can be adapted to receive a treatment location from the medical encounter computer system and generate a treatment location notification according to a comparison of the treatment location and the medical regulatory record. The set of computer readable instructions can be adapted to receive a healthcare provider arrival time, receive a healthcare provider departure time, calculate an on-location time, and generate a temporal notification according to a comparison of the on-location time and the healthcare activity information. The set of computer readable instructions can be adapted to receive a healthcare provider arrival time, receive a healthcare provider departure time, calculate an on-location time, and generate a temporal notification according to a comparison of the on-location time and the medical regulatory record. The healthcare activity information can be a first healthcare activity information included in a set of healthcare activity information stored on the immutable storage system and the set of computer readable instructions are adapted for creating a token representing a portion of the set of healthcare activity information and providing the token to a third party. The set of computer readable instructions can be adapted for effectuating a medical transaction according to the healthcare activity information. The medical transaction can be the effectuation of a smart contract. The medical transaction can be placed in a queue for approval prior to its effectuation. The set of computer readable instructions can be adapted to retrieve warranty information for a medical device used in the medical activity and generate a warranty notification according to a comparison of the warranty information and the healthcare provider identifying information.

### BRIEF DESCRIPTION OF THE DRAWINGS

FIGS. 1A-1E shows various side views of aspects of the system.

FIG. 1F is a block diagram of components of aspects of the system.

FIG. 2A is a schematic of aspects of the system.

FIG. 2B is a schematic of aspects of the system.

FIG. 2C is a schematic of aspects of the system.

FIG. 2D is a schematic of aspects of the system.

FIG. 3 shows a flowchart illustrating aspects of the system.

FIG. 4 shows a flowchart illustrating aspects of the system.

FIG. 5 is a schematic of aspects of the system.

FIGS. 6-8 are flowcharts illustrating aspects of the system.

FIG. 9A shows a plan view of a treatment location.

FIG. 9B shows a plan view with geofencing at a treatment location.

FIGS. 10A-10B show flowcharts of aspects of the system.

FIGS. 11-18 show flowcharts of aspects of the system.

FIGS. 19-20 show schematics of aspects of the system.

FIG. 21 shows a flowchart of aspects of the system.

### DETAILED DESCRIPTION

The present system provides for real time verified pairing of medical activities, individuals, events, and objects including physical articles or tasks such as medical research, medical treatments, a medical device, testing, clinical trials, and a medication with a virtual (e.g., digital) representation. The computer system can be a kiosk, mobile device or otherwise, and can include a set of computer readable instructions configured to receive information about an article or task that can include the ability to capture information such as research, description of the article, task or process, manufacturer, characteristics of the medical activity, article, task, process, procedures, materials, object and the like, an event associated with the article or task, its significance, the individuals associated with the article or task such as researchers, shippers, manufacturers, distributors, medical service suppliers, dispensaries, medical facilities, date and time, location, individuals that can verify or certify the generation of the digital representation and the meta data associated with the medical activity. The computer system can be a mobile device.

The information that can be inputted, received by the sensor, retrieved from a computer device or immutable storage system can include a number of data elements. Data can be retrieved from a medical device such as one that reads vital signs, information about medical treatments, the patient, medical apparatus such as blood pressure monitors, thermometers, and other devices such as dialysis machines, x-rays, CAT scan machines, MRI machines, and the like. A medical device computer system can be used to receive information from these sources as well as a database, local and remote, and a sensor. The sensor can include video, audio, electrical signals such as RF, Bluetooth, and other wireless devices as well as wired devices such as a thermometer attached to the medical encounter computer system.

Data can include a healthcare provider record that can include information about a healthcare provider such as name, licenses, authorizations, skill, assignments, work schedule, privileges, and the like. Authorizations can include what medical activities and medications that the medical provider can administer. For example, a surgeon may require an orthopedic license or authorization to perform orthopedic surgeries. A patient record can include patient information such as names, address, location, treatment locations, diagnosis, treatment plans, insurance, approved providers, and the like. A set of computer readable computer instructions can be adapted to retrieve such information, and generate notifications based upon a comparison of the various data it receives. A notification can have several forms such as generating an approval, a warning, a block to prevent the healthcare activity, and the like. For example, the computer readable instructions can compare the usage of healthcare provider licenses required for use of a medical device with the licenses of the healthcare provider and, if the health care provider has the requisite licenses, provide a notification that the healthcare activity is approved. If the healthcare provider does not have the requisite licenses, then the computer readable instructions can generate a warning that the activity is not approved and in one embodiment, prevent the medical device from operating.

The sensor can also receive biometric information of the patient and the healthcare provider so that the identity of the patient and healthcare provider can be known to the medical encounter computer system. The system can also identify the location of the medical device and the healthcare provider and compare the two. In the event that these locations do not match, it can indicate that incorrect information is being entered associated with a medical activity. For example, if there is an attempt to enter a medical activity for reimbursement purposes and the health care provider location and the patient location do not match, it can indicate a mistake or fraud. In this case, the health care activity may not have occurred as the patient and the healthcare provider were not in the same location. The same comparison can be performed with the medical device location and the patient location. The medical device can include location information that can be received by the computer readable instructions. The same comparison can be performed with the healthcare provider location and the medical encounter computer system. The same comparison can be performed with the patient location medical encounter computer system. An approved patient location can be included in the patient record which can represent a location where the patient is to receive healthcare treatment. The patient location can include one or more locations such as the patient in home treatment location, in patient location, outpatient location, and the like. In the event that the comparison of these data points do not match, the computer readable instructions can create a notification that warns that a mistake or even fraud occurred, flag a record, event or set of records or events for review or audit, determine that the healthcare activity is not approved, determine that the healthcare activity should not be or have been performed, and the like. The system can also receive and use the biometric information of patient, biometric information of a health care provider and determine a time of activity or event to confirm a patient encounter.

The system can also receive health care activity from the sensor or other input that can include use of a medical device, use of an external sensor, administration of medication, application of a medical device, medical treatment, and the like. For example, the sensor can detect physical therapy treatment and activity and can determine if the patient was to receive such treatment. Through the detection of input from a healthcare provider and recording of the motion associated with physical therapy, the system can determine if the treatment was to be administered according to the patient record, healthcare provider, record, insurance information, treatment plan, and the like. The insurance information and treatment plan can be in the patient record or in a third-party database that is associated with the patient and healthcare provider.

In one embodiment, the system can determine travel time of a healthcare provider, medical device, and patient from one location to another. The system can determine an arrival time and a departure time of the healthcare provider. The system can have information concerning the healthcare activity and compare a time period associated with a healthcare activity with the time that the healthcare provider was at the patient location and create a temporal notification according to the comparison. For example, if the health care provider was to take a sample of blood, a fifteen-minute time period on location may be reasonable. If the health care provider was to administer physical therapy, a thirty-minute time period on location may be reasonable. If the on-location time was for fifty minutes, it may not be reasonable. The system can also determine a travel time from one location to another. The system can determine a departure time from a first location (e.g., first patient) and an arrival time to a second location (e.g., second patient) to determine a travel time wherein the locations can be within a facility of between two facilities (e.g., one room to another or home to home). If a second health care activity occurred prior to the ability of the health care provider to reasonably travel from the first location to the second location, a notification can be generated. In one embodiment, the system can calculate the travel time according to a mode of transportation that can include walking, biking, motorized vehicle, public transportation, and the like.

When administering medical, the system can retrieve information from a medication computer system. A medication record can include information about medications that are approved for the patient, acceptable substitutes, drug interactions, insurance approved medications, and the like. The system can generate a notification according to the comparison of the medication and patient record and patient identification information. For example, if the patient is not to receive a certain medication and the system determines that this medication is being administered, a warning can be generated. If the medication is not approved for insurance reimbursement, a warning can be generated. If the medication needs to be administered according to certain regulations or healthcare provider authorization, and is not, a warning can be generated. If the patient physiology is outside acceptable ranges, a warning can be generated. The information that is received and generated by the medical encounter system can be recorded on an immutable storage system. In this configuration, a token can be generated that points to one or more components of the medical record and its metadata or one or more portion of the medical record. In one embodiment, smart contracts of other auto initiating software or processes can be included in the system for adding data, hashes, blocks of other items or data to a storage system or cause a computer system to implement a routine, transaction or process. For example, if the patient needs verification that a healthcare provider visit occurred, the token can be associated with the date, time, and location of the medical visit without revealing the underlying activity, such as the treatment, diagnosis, and the like. In one embodiment, the token can be a non-fungible token. Therefore, verification that the patient can provide proof that medical treatment was received with disclosing personal medical information. Such application can be useful for excused absences from work and school. The system can also be used to effectuate a smart contract that can be associated with payments. When the system determines that an appropriate medical activity occurred, the system can instigate payment from an insurance company to a health care provider. The system can also place the payment in a queue for subsequent review so that the payment can be automatically generated, but human intervention for approval can still be accomplished.

The system can also facilitate warranty claims for such items as medical devices. For example, a medical device manufacturer may require that the patient have or not have certain attributes (e.g., under a certain age, weight, blood pressure, insulin levels, etc.) or that the medical device only be installed by an authored (e.g., licensed) medical professional. The system can make this determination and store the information in the immutable storage system for later retrieval, in the event that the information is need for a warranty claim, and the like.

For example, the system can capture an image of the individual, item, activity, and its unique identifier and associate that information with a date, time, and location. Individuals can be captured that can include images, video, date, time, and location information. The system can retrieve scheduling and occupancy information and verify that the individuals involved were at the same location at the stated date and time. Images of the article or task can be compared with environmental information (e.g., weather) information at the location, date, and time so that the background of an image showing the environmental conditions can be compared with third-party weather information. If the image of the article shows a cloudy day and the third-party weather information shows a cloudy day, the confidence of the virtual representations of the article increases. Biometric data can be captured from one or more individuals as well as attendance information from access control system to verify that the individual was at the treatment location at the date and time the virtual representation is retrieved, created, and stored. Further, the information can show that a first medical provider encountered a patient at a first date, time, and location and a second medical provided encountered the patient as a second date and time, and same location increasing the confidence that the medical activity properly occurred. The confidence score can represent the probability that the physical individual, action, and objects match the digital representations can be derived from a number of data points. For example, if a patient first location at a first time is 100 miles away from a patient second location at a second time and the difference between the two times is 15 minutes, the confidence is low. This determination can depend upon the travel mode as well.

The information captured can be stored in a record that include fields associated with the above as well as individuals involved, observers, materials used, equipment used, processes used, location, date, time, results, class, type, lot, batch, or other identification of the article or task, one or more sources, cost, care instructions, material specifications, shipping information, storage information, and other information. The medical object record may be created or updated as actual articles arrive to a location and have serial numbers, bar codes, QR codes, RFID values, beacons, lots, sizes, or other components or material identification added or associated with the article record. A medical activity record can include a task information associated with article such as research, reactions, processes, procedures, manufacturing, packaging, shipping, storage, testing, trials, and any combination.

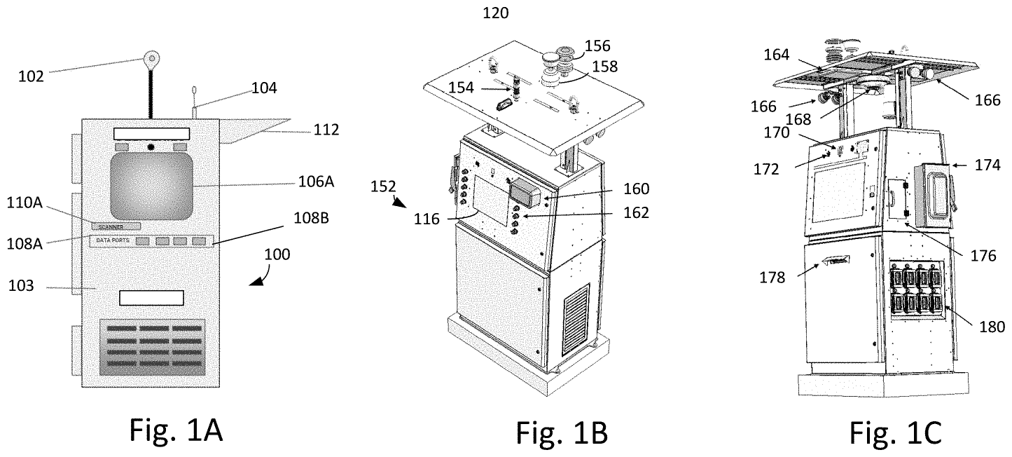

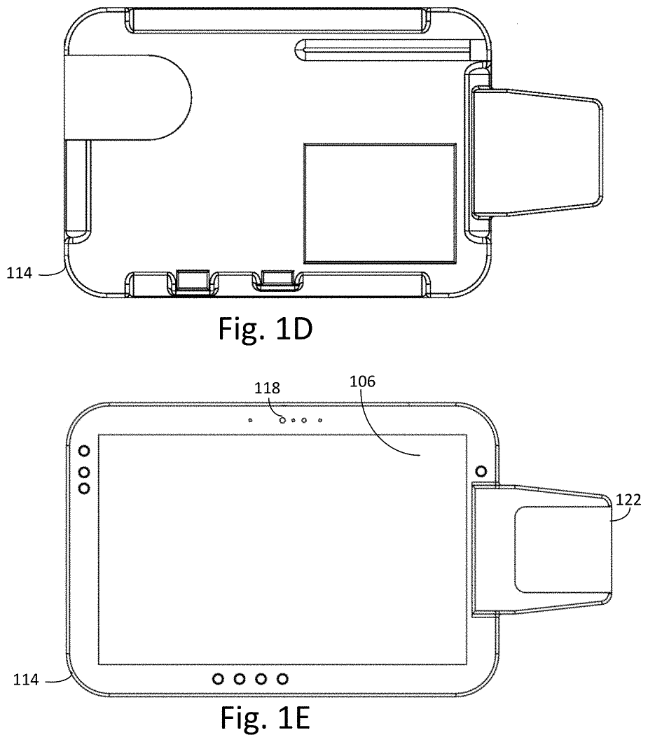

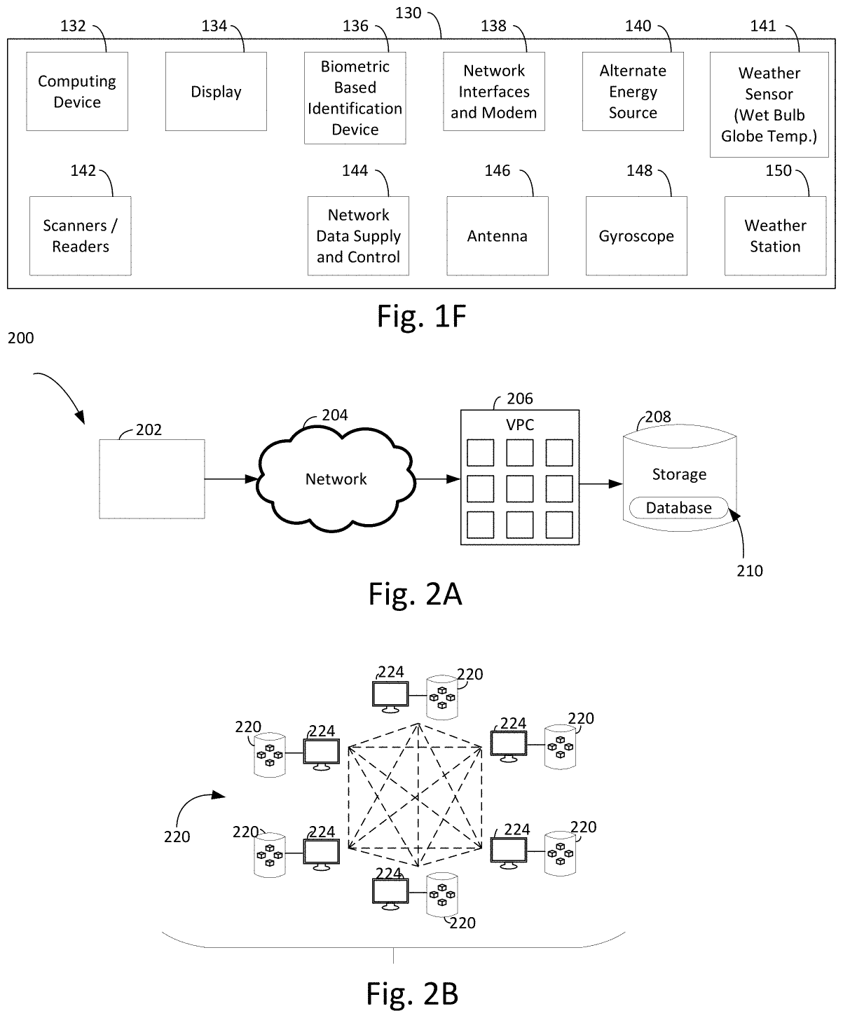

FIGS. 1A through 1D, they illustrate one embodiment of a system 100 that can be a computer system in a kiosk, or other housing, that can be uniquely associated with a treatment location. The computer system can also be removably connected to the housing and can be a portable computer device. If the computer device having the controller is a portable computer device, the portable computer device can be physically attached to a treatment location, virtually associated with a treatment location, or can be identified as being at a treatment location (e.g., using global positioning coordinates) when data is captured with the computer device so that the date, time, and location can be associated at the time of capture.

The system can be implemented as a kiosk that can be mobile and include a housing having a controller. The housing 103 may be located at a treatment location and include a controller in communications with a computer readable medium. The housing can be physically associated with the treatment location, virtually associated with the treatment location, or both. A location marker can be affixed to the treatment location such as in a placard, label, or otherwise affixed at the treatment location. The housing can be removeable and attached to the treatment location so that it is stationary during a first task process but can be moved to a second task or process at a different physical location once the first task or process is completed. For example, the housing can be positioned at a first patient room, a medical testing area, and a second patient room. The housing can be on wheels or other mobile assembly allowing the housing to be moved from location to location.

For a location marker, a transmitter such as a RFID can be associated with the treatment location by embedding it is a permanent fixture such as a concrete slab, foundation, structure, and the like. The system can read the information from the location marker and associate its actual location with the treatment location. The location marker can include an alpha, numeric, or graphical information such as a number, letters, barcodes, QR code, physical or geographic coordinates (e.g., GPS coordinates), passive transmitter, active transmitter, and the like. Each system can have a unique identifier and each treatment location can have a unique identifier.

FIG. 1A shows a first side of the system 100. The system 100 can include a camera 102 for obtaining images of articles, individuals, or other items at entering or leaving the treatment location, as well as images of individuals along a perimeter. The camera 102 may capture biometric images upon which biometric recognition may be performed. Multiple cameras may be placed on or around the housing. The cameras may have biometric recognition and motion detection capabilities. System 100 may include an addition to the camera 102 or instead of the camera 102, biometric-based identification devices that may be used to confirm the identity of individuals entering, leaving or on the perimeter of the treatment location. The system 100 may include an antenna 104 for communicating with a network including a wireless network, Wi-Fi network, Bluetooth, quantum networks, cellular network (e.g., 4G or 5G network), and any combination. The system can be a wired network that can include wireless components. The system 100 may include a housing 103 made of suitable weather resistant material, appropriately sealed to protect the internal hardware. The system 100 may include a display 106A, such as a touchscreen display, upon which information may be displayed and entered. The display 106A may include an integrated camera that may be used to capture images and that may be used in performing facial recognition of individuals and other material. The display may also include, or be operatively associated with one or more integrated speakers for providing audio output, a microphone for receiving audio information to facilitate two-way communications to a remote location. The system 100 may include a scanner 110A for scanning items, such as devices and medications, as will be explained in more detail below. The scanner 110a may be, for example, a QR scanner, an Optical Character Recognition (OCR) or a bar code scanner 110A in some instances. The side of the system 100 shown in FIG. 1A can be used for deliveries and dispensaries. A delivery person may scan delivered materials, equipment, medical devices and components, medicines, or other items via the scanner 110A and may interface with the system using the touch screen display 106A. An individual may scan or take images of items related to an inspection such as inspection documents via the scanner 110A or camera and may interface with the system using the touch screen display 106A. In some embodiments, there may be fewer sides in which to interact with the system for all authorized personnel. In some embodiments, the controller or kiosk can be wall mounted.

The housing can include information displayed such as notes, reports, results, status of task, and the like. In some embodiments, the information displayed can be in electronic form so that a video display is provided. In one embodiment, the unique identifier associated with the treatment location may be displayed. Other location identifying information can be displayed such as location number, medical provider license or certification number, treatment location, and the like. In addition, the site address may be displayed and may refer to both the mailing address for the treatment location and other physically identifying information associated with the location.

Referring to FIGS. 1B and 1C, the housing 152 can include a side that is configured to be used by an individual at the treatment location. The housing can include an alarm indicator 154 that can be actuated as described herein. The housing can include input for receiving information from third party sources such as testing equipment, medical equipment, access control systems, accounting systems, workflow automation systems, quality control systems, scheduling systems, and the like. Biometric reader 160 can include an iris scanner, fingerprint scanner, palm print scanner, facial scanner, DNA, phlebolic confirmation or some combination. Display 116 can be proximity to input assemblies such as physical or contactless buttons 162 of facial recognition or other sensors. The housing can include a field receiver 164, lights 166 and camera 168. One or more cameras can provide a 360° field of view and include a wireless connection for transmitting images to a remote computer device. The images can also be used for input to the system including input allowing the system to identify delivered tasks and materials. The system can include one or more second cameras 170 such as webcams disposed at various locations around the system for capturing images. The lights can include motion activation and photoelectric activation. Speakers 172 can be included to provide audio information to a user using—or near to—the system. The audio information can include instructions, alarms, and the like. Power junction 174 can include a shut off switch that can be used in emergency and non-emergency situations. The power junction 180 can be used for providing power to system including medical equipment, diagnosis equipment, and the like. The system can include a secondary power source, such as a battery, so that when the main power is shut off, an alarm can sound, and a notification can be sent to a remote computer device indicating that the system or power source has been powered down. The system can include a hand scanner (not shown) that can be protected by a hand scanner access door 176. A document scanner 178 can be included in the system for receiving physical documents, converting the physical document into a digital representation, and storing the digital representation on the computer readable medium or the immutable storage system. The system can include a wired connection to remote computer devices of a transceiver to provide a wireless connection to remote computer devices, including medical equipment.

Referring to FIGS. 1D and 1E, the housing can be a mobile housing 114 and can include a display 106, microphone 118 and sensor 122. FIG. 1F depicts components that may be included in the system of exemplary embodiments even when not included in a housing. The system may include a computing device 132. The computing device 132 may take many different forms indicating a desktop computing device, a laptop computing device, a mobile computing device, an embedded system, a smartphone, special computer device, custom computer device, or the like. A display 134 may be integrated with the computing device 132 or as a separate device, such as a liquid crystal display (LCD) device, a light emitting diode (LED) display device or other types of display devices on which computer information may be displayed. One or more biometric-based identification devices 136 may be provided. Multiple biometric-based identification devices may be used. Network interfaces and a modem 138 may be provided. The network interfaces may interface the computing device 132 with a local area network or a wide area network wherein the networks may be wired or wireless. A modem may be provided to communicate telephonically or over cable lines with remote computing devices.

The system 130 may be implemented in a distributed fashion and may include an alternative energy source 140. For example, solar panels, wind turbine(s), a battery, or the like may be used. For example, solar panels or a cable to a wind power source could be configured to provide power to the system and can be affixed to the system or housing. Alternatively, a power line leading to the alternative energy source may be connected to the housing and system to provide power to the system, housing, and associated components, such as external power supplies.

The system 130 may include various scanners and readers 142, such as those described above relative to the housing. The system 130 may include an internet data supply control 144 and a mechanism for turning the access to this service on and off under a programmatic control. Programmatic control may be provided to grant or deny access to such resources including medical equipment, storage areas, and diagnostic equipment. The system 130 may include an antenna 146 for wireless communications signals to receive and transmit. The system 130 may include a gyroscope 148 to monitor any moving of the system. The gyroscope 148 may indicate motion indicative of whether someone is trying to move or tilt the housing or other component of the system. Logic may be provided to send a notification in such an event where the gyroscope indicates substantial enough movement. The system 130 may include a weather or environmental station 150 to measure current environmental conditions, such as temperature, air movement, humidity, precipitation, barometric pressure, direct sunlight, and the like. Input from the station 150 may be used to inform decision making by the system in some instances. Alternatively, the environmental conditions may be collected via software, such as from a weather service, building system, air conditioner, or other sources. Similarly, the system 130 may include a fluid sensor 141.

The computer system can be a portable system that can be generally handheld or disposed on a rolling cart. The computer system can be in a housing that can be affixed to a treatment location. The computer system can be mobile so that it can be moved from treatment location to treatment location, such as with a mobile testing facility.

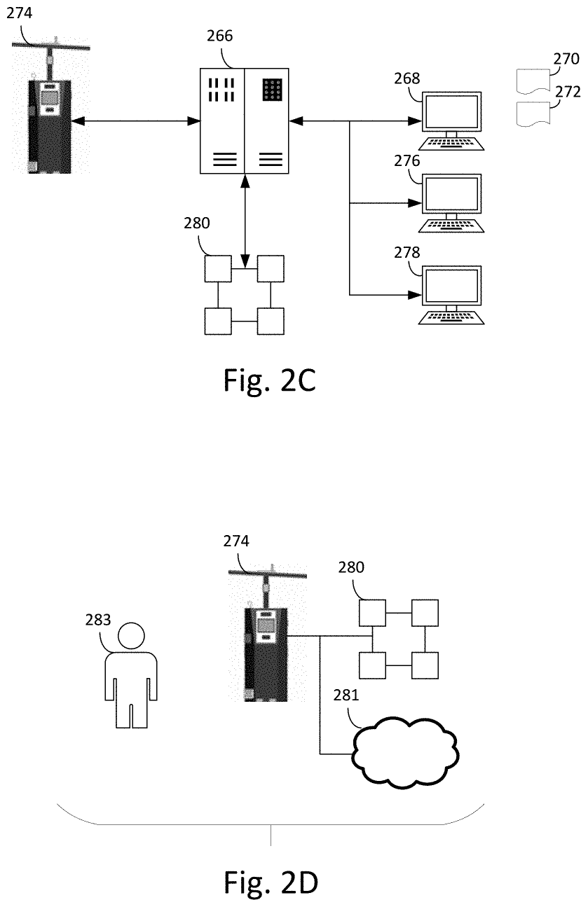

As shown in FIG. 2A, the exemplary embodiments may be implemented in a decentralized computing environment 200, that may include distributed systems and cloud computing. FIG. 2A shows one or more systems 202 that may be in communication with a remote cluster 206 via a network 204. The cluster 206 may store information received from the system 202 and provide added computational functionality. The network may be a wired network, a wireless network, or a combination thereof. The network 204 may be a secure internet connection extending between the system 202 and the cluster 206, such as a virtual private cloud (VPC). The cluster 206 may include access to storage 208. The storage 208 may include a database 210 in which information regarding a treatment location is stored in a consistent manner.

FIG. 2B shows diagram 220 of an example of a peer-based network where an immutable storage system includes the ability to broadcast data and provide shared among the nodes 224. This network may be resident in the VPC cluster 206 (FIG. 2A) or in the network 204, for example. The nodes 224 may represent computing resources, such as server computer systems or other computing systems with storage devices 220. FIG. 2C shows a kiosk 274 in communications with a server 266 that can be in communications with a distributed network 280 or computer, storage devices, or any combination. Third party computer system 268, 276 and 278 can be in communications with the server 266 and kiosk 274 so that information 270 and 272 can be shared with these systems. FIG. 2D shows a user 283 using kiosk 274 (stationary or floating) to access information from distributed storage 280 as well as transmit and receive data from a global communication network 281.

The information from a treatment location, medical provider, research, lab, manufacturer, supply company or other third party can also be stored on the immutable storage system and retrieved by the system. The computer system can be configured for receiving a medical device record or a medicine record from the originator representing the medical device or medicine, creating an actual medical device or medicine requirement record representing the actual article delivered to the treatment location, creating a final medical device or medicine record (e.g., medical object record) according to a difference between the article requirement record and the article material record, receiving receipt verification information representing the actual article was received by an authorized individual 283 (FIG. 2D).

Processes, projects, and task specifications, which may be needed for compliance with warranty, insurance, design, specifications, inspection, and other requirements, can be received at 276 and requirements can be received from a requirements computer device 278 either directly or from the immutable storage system. The requirements can include approved materials that are approved by regulatory entities, such as governments, researchers, manufacturers, technicians, healthcare workers, distributors, designers, and the like. Requirements can include specifications, materials, safety codes, and individual licenses, and the like. The requirements can include processes and procedures for handling, use, installation, and assembly of the medical device or medicine.

The various computer devices, including the server and remote computer device (e.g., system, controller, and any combination), can be in communications with immutable storage system 280. The immutable storage system can include a distributed ledger, immutable database, block-chain structure, and the like. The communications between the various computer devices, including the server, the remote computer device, and immutable storage system can be a global communications network, wide area network, or local area network, delivered to a computer readable medium from one device to another (e.g., USB drive, CD, DVD) and can be wired or wireless.

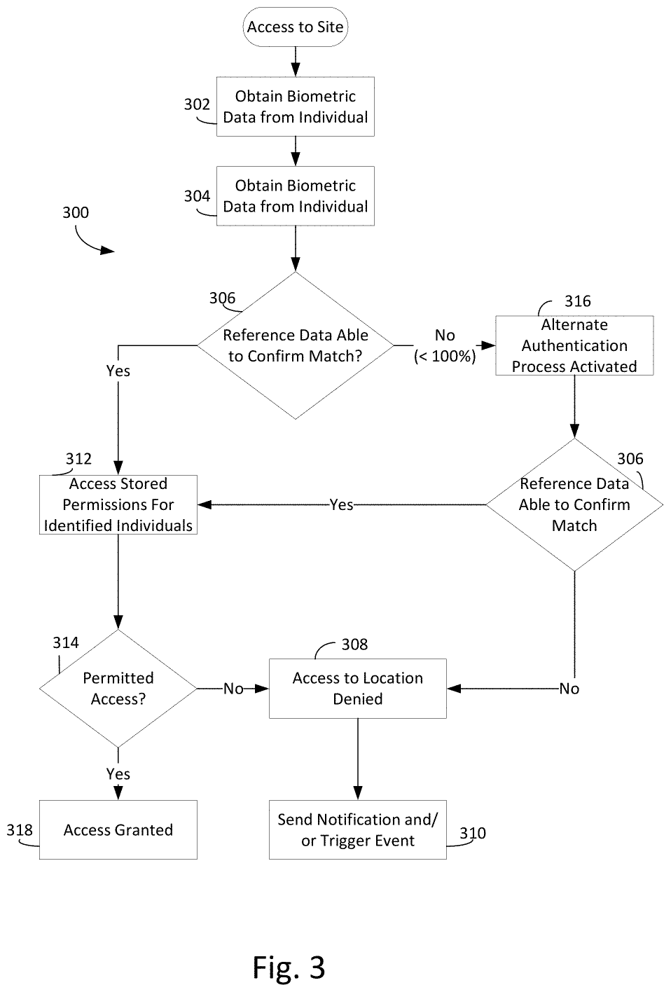

FIG. 3 shows a flowchart 300 identifying steps that may be performed in exemplary embodiments regarding this functionality of the system. Initially, biometric data is obtained from an individual, such as a medical provider or patient, or other individual that is seeking access to the treatment location 302. In some exemplary embodiments, a camera 102 may capture an image of an individual and facial recognition may be performed. The biometric data in one case is the facial image of the individual. In other exemplary embodiments, the biometric data may be, for example, fingerprint data, hand scan data, voice print data, retinal scan data or the like, gathered by appropriate biometric-based identification devices. The obtained biometric data is stored, and then previously stored data is accessed from storage to compare biometric data for known individuals and to attempt to identify the individual 304. A comparison may be made between the gathered biometric data and the known biometric data to determine if there is sufficient closeness for there to be a match. Information regarding the identity of the individuals for which the biometric data is stored is also stored in the storage device. A determination is then made whether there is a match or not 306. An individual data can include the credentials of the individual and used to compare the access attempt with the record associated with the individual.

If there is not a match 306, a manual process may be executed, or an alternative authentication process may be deployed 316. If this alternative authentication fails to produce a match 306, access to the treatment location may be denied 308. In addition, a notification may be sent to a responsible party and an event may be triggered, such as contacting security or law enforcement officials 310 such as in the case of an attempted theft of medical devices or medications. If the alternative authentication process produces a match, the process proceeds to 312.

The system may store permissions for anyone allowed to access system and treatment locations. These permissions may identify the dates and times where the individual is given access to the treatment locations, the system, or portions of the system. In addition, the permissions may specify what materials, articles, and tasks the individual can access or preform. These permissions may be accessed to determine the permissions for the identified individual 312. If the permissions indicate that access is permitted 314, the individual may be granted access to the treatment location and article at 318.

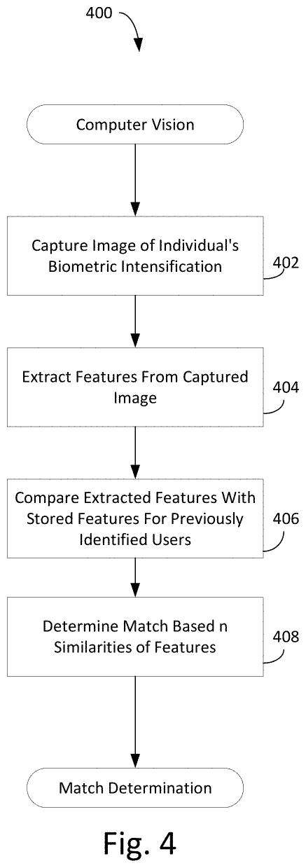

FIG. 4 shows steps that are performed in a case of computer vision for 302, 304, and 306 of FIG. 3. The flowchart 400 begins with 402 in which an image of an individual is captured for biometric recognition. This may be captured by a number of different types of image capture devices, including an intermittent video camera, still camera, iris scanner, facial scanner, fingerprint scanner, or other type of capture device. In the case where an image of the face of an individual is captured, identifying features may be extracted from the captured image 404. In other words, unique facial features that help to identify an individual are extracted from the image. The image may be filtered and normalized. The features are then compared with the stored features for identified individuals 406, determination is made whether there is enough similarity for there to be a match.

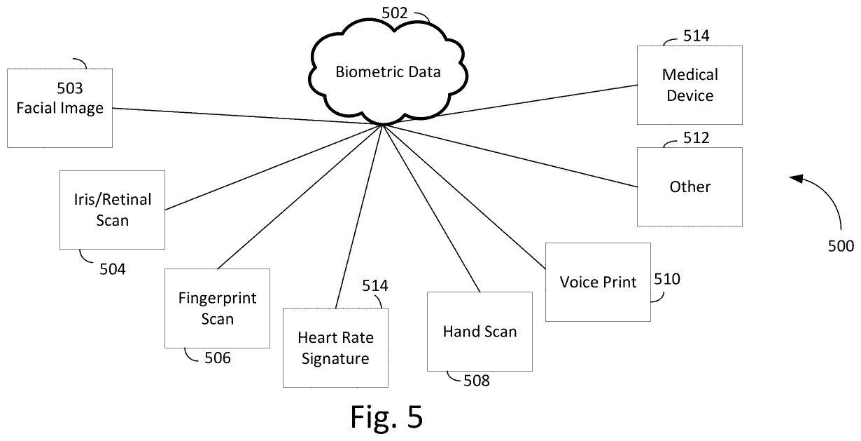

FIG. 5 shows a diagram 500 that illustrates various types of biometric data 502 that may be obtained by biometric-based identification devices at the treatment location to attempt to identify individuals. Biometric data may include facial recognition 503, an iris/retinal scan 504, a fingerprint scan 508, a hand scan 508, a voice print 510, or heart rate signature 514. It should be noted that other types 512 of biometric data may also be used in exemplary embodiments to help identify individuals uniquely. Also, an individual may be required to provide multiple types of biometric data in some instances. In one embodiment, the biometric data or patient identification information can include information that is associated with a medical device. For example, medical devices can include unique information. Pacemakers and orthopedic prostheses include one or more serial numbers that can be associated with barcoded labels and can be associated with a patent and appeal in medical records. Therefore, the patient can be rapidly identified using the medical device. The sensor can read the information, even when the medical device is implanted, retrieve the associated patient information from a patient record and identify the patient. The sensor can read the information from a medical device though wireless communications with the medical device. The medical device can provide the information directed or a RFID can be associated with the medical device and attached to or even implanted with the medical device.

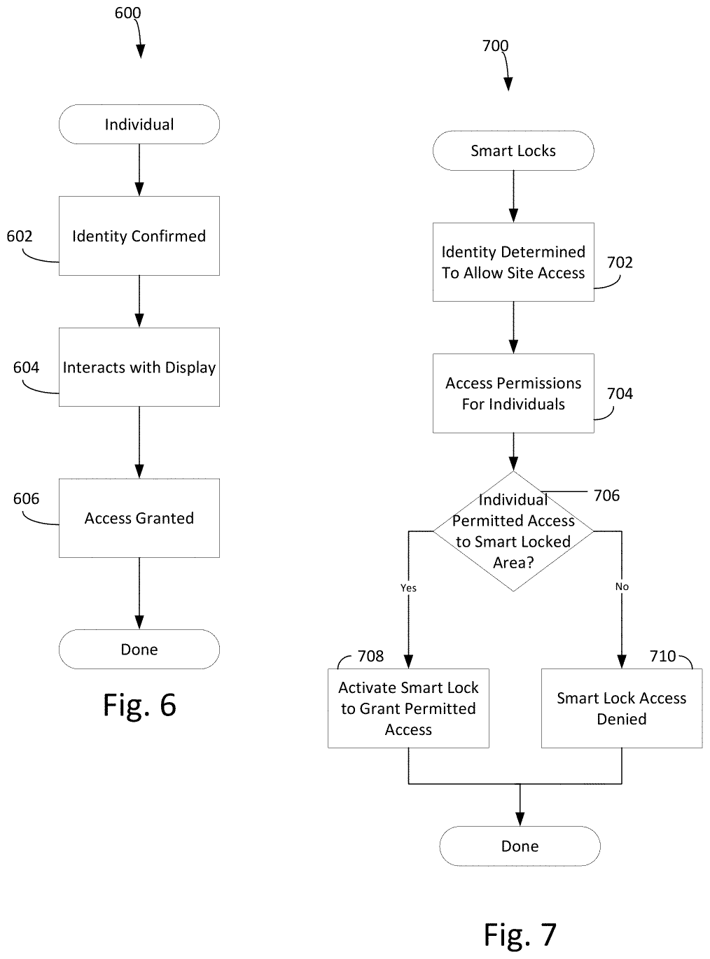

FIG. 6 shows a flowchart of the steps that may be performed to ensure that an individual gains access to the appropriate items once they have been granted access to the treatment location. As shown in the flowchart 600 of FIG. 6, initially the individual has their identity confirmed, as has been discussed above 602. The system may offer an alternative touchscreen option to place a call to the appropriate party should the software fail to verify and otherwise authorized the individual. The individual may be prompted to interact with the display, such as the touchscreen 1068 (FIG. 1) to register and to indicate whether they seek certain items. For example, with the user interface, the individual may activate the individual activatable elements. Access is then granted at 606.

FIG. 7 provides a flowchart 700 of the steps that may be performed relative to smart locks at the treatment location that can control access to areas, storage, devices, and the like. The individual has his/her identity determined and has access to the treatment location 702. The system may offer an alternative touchscreen option to place a call to the appropriate entity or location should the software fail to verify an otherwise authorized individual. The permissions stored for the individual are accessed 704. A determination is made whether the individual is granted access to a smart locked area 706. If the individual has permission to access the area 708, the smart locks may be de-activated to unlock the area, storage location or other place such as where articles including medical device, medication, and materials are located. If the individual lacks the proper permissions to access the article or location, then access can be denied 710, such as by keeping the smart locks locked.

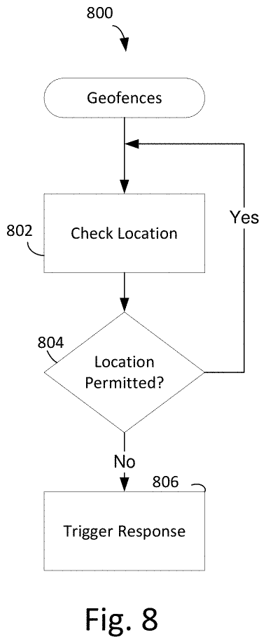

As shown in FIG. 8, a flowchart 800 shows some of the steps performed automatically and can be performed without notification to an individual. The process begins with the checking in of the location 802 of an individual. A determination is made in 804 whether the individual is permitted to be at that location. If the individual is not permitted to be at that location, a response is triggered 806.

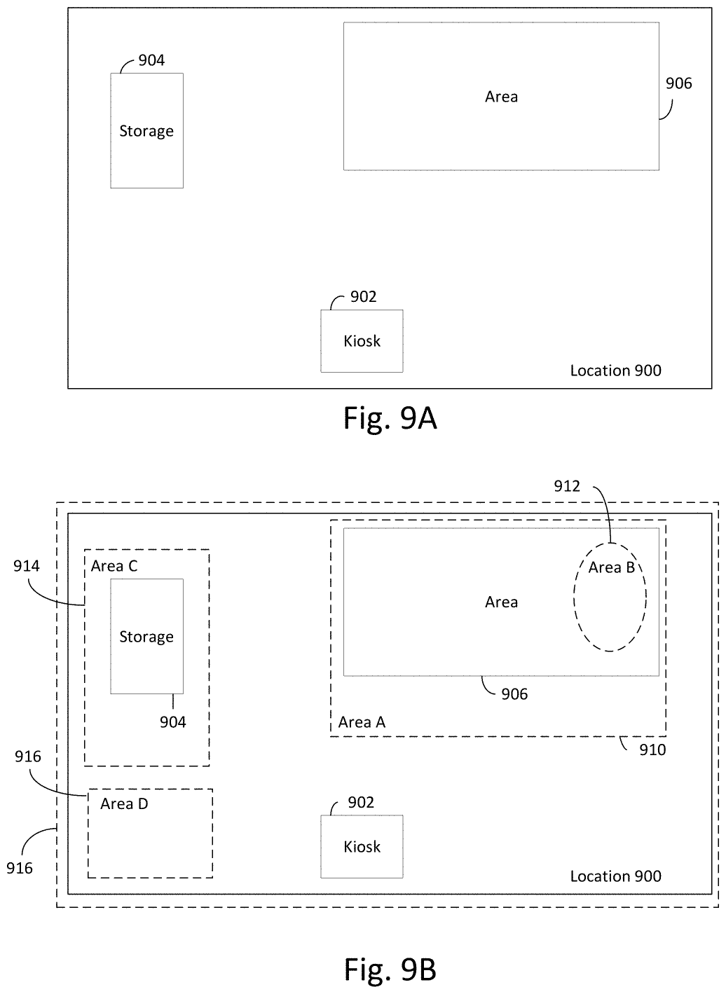

To assist with illustrating geofencing, referring to FIG. 9A, it shows an illustrative treatment location 900. The treatment location 900 may include a housing 902 for the system as well as storage location 904 that can be a building, treatment locations, mobile clinic, or the like. The storage location 904 may hold articles which can include medical devices, medicines, containers, instructions, manual, and the like. The treatment location 900 may also include a medical activity location 906. The medical activity location may be where tasks are performed using materials that can result in a medical device or medication being administered.

FIG. 9B shows an example of different areas that may be established for geofencing at the treatment location 900. Area A shows a boundary 910 that may include the entirety of a certain treatment location 906 (e.g., medication storage and research laboratory). Area B 912 may be a portion of the treatment location, such as where medical devices or medicines are stored. Area C 914 may be another location and Area D 916 may be the entire treatment location. Individuals may have access to none of these areas, to a subset of these areas, or all areas.

When an individual enters an unauthorized area, one option is to send a notification, such as a phone call, a text message, an email message, or other notification to a supervisor on or off the treatment location. Yet another option is to take disabling measures relative to an individual attempting to access the area, perform an unauthorized or unlicensed action, administer a medication without authorization of in conflict with other controls, and the like. The individual can be a healthcare worker, supplier, administrator, and the like. The disabling measures could entail triggering locks, disabling equipment by shutting off power, or the like. The geofencing can also be used to determine where the material will be delivered, stored, and installed. The geofencing, along with date and time information, can assist with the determination whether the material was handled or installed by a licensed, experienced, approved, authorized, or otherwise preferred individual. Further, items, including high value items, can be tracked by the computer system described herein and can be paired with digital representations and verifications.

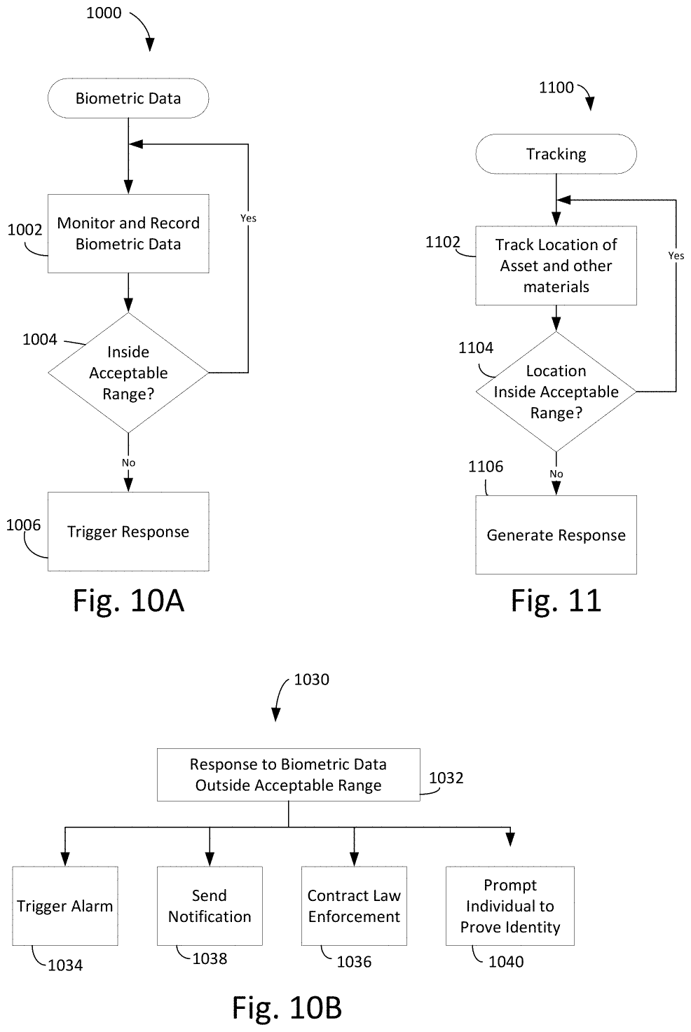

Referring to FIG. 10A, biometric data is captured and recorded as 1002. If the individual associated with the biometric data is not approved to be within an area at 1004, a response is triggered at 1006. Referring to FIG. 10B, if the biometric data shows that an individual is outside an acceptable range, an alarm can be triggered at 1032, notification of the alarm can be sent at 1038, law enforcement can be contacted at 1036 and the individual can be promoted to prove their identify at 1040.

Referring to FIG. 11, the system may track at 1102 the medical device, medication, or other article at the treatment location The system can check whether the location of the medical article, medical device, other medical materials is acceptable or not 1104. For example, suppose that pharmaceuticals have been delivered to the treatment location. Subsequently, the pharmaceuticals is removed from the location without proper authorization which is problematic and would warrant a response such as a warning or notification. If the action at the location is not acceptable as checked in 1104, a notification can be generated in 1106.

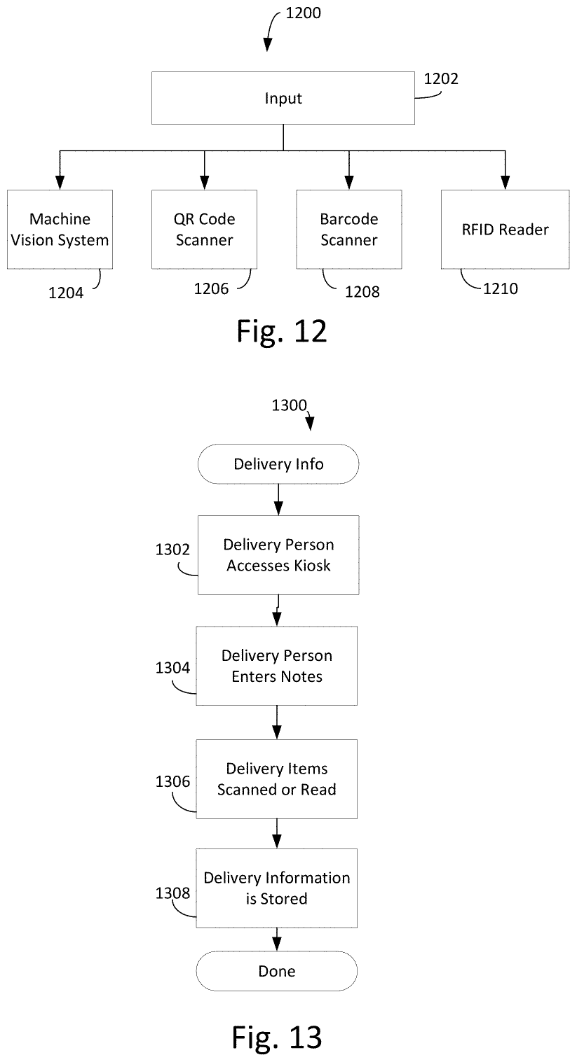

Diagram 1200 in FIG. 12 illustrates different types of inputs 1202 that may be used for assisting gathering information regarding deliveries. A machine vision system 1204 may be provided. The machine vision system 1204 may capture an image of the delivered items and process the image to determine the nature of the items that were delivered as well as the quantity of items. Moreover, the machine vision system may capture an archival image that may be indicative of the state of the items when they were delivered. A QR code scanner 1206 may be used where QR codes are on a delivered items or documentation. Similarly, a bar code scanner 1208 may be used where bar codes are on the items or on documentation delivered with the items. Still further, an RFID reader 1210 may be provided to gather information regarding the delivered items.

The delivery person may interface with housing via display 106A and 1900 to provide delivery information. Flowchart 1300 of FIG. 13 illustrates some of the steps that may be performed in such an instance. Initially, the delivery person may access the housing 1302. The delivery person may enter a note(s) regarding the delivery, such as what was delivered and the state of the items that were delivered 1304. This information may be entered, such as through the display 106A (FIG. 1A) which can be a touchscreen. The delivered items are imaged, scanned or read 1306. As was mentioned above, several different types of input technology may be used on the delivered items. Therefore, delivery information is then stored in records that may be accessed subsequently 1308.

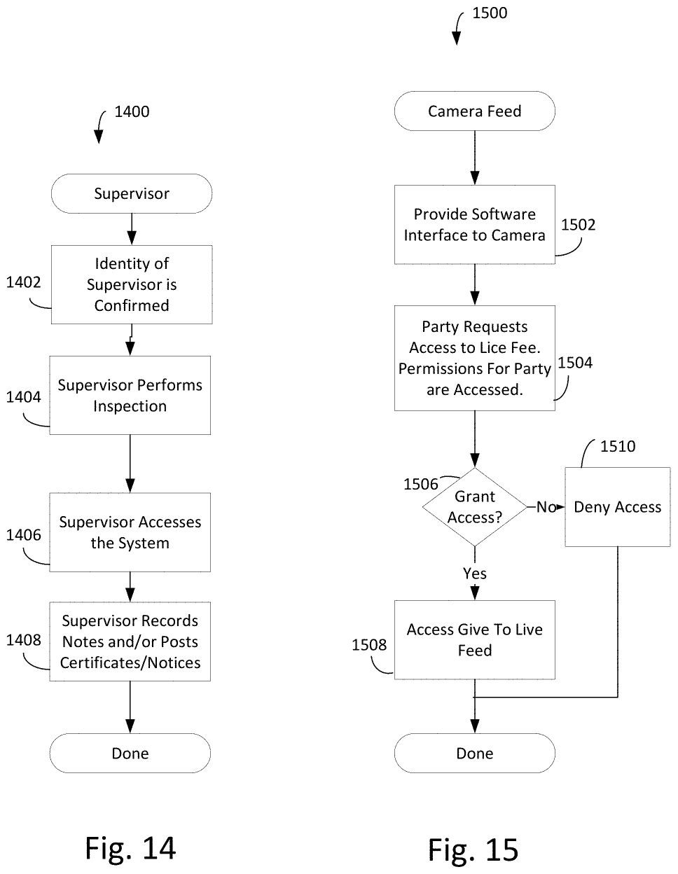

When a transaction, other than an original entry of an article, a subsequent transaction, an individual (e.g., researcher, manufacturer, technician, medical provider, healthcare worker, custodian, certifier, inspector, supervisor, and the like) may interface with the system. FIG. 14 includes a flowchart 1400 illustrating steps that may be performed in such an interaction. Initially, the identity of the individual, a supervisor in this example, may be confirmed using the biometric data 1402 or manually using the touchscreen on the system. The supervisor then performs the inspection of medical activity at the treatment location 1404. The supervisor then accesses the system at 1406 and provides information about the activity as the system is reading information about the activity. The supervisor then may record notes and post certificates, notices, or other information at the system 1408. Additionally, the supervisor may use technology available via the system such as OCR scanner, camera, or the like to capture appropriate information the individual may include during the recording of the article or transaction. If the system is at a medical treatment, for example, the metadata about the activity, medical device or medicines can be recorded.

The system may include a still camera(s) or a video camera(s) that can be included in the system. FIG. 15 provides a flowchart 1500 relating to such access. A software interface to the camera may be provided to enable authorized external parties to gain access to the camera 1502. A party requests access to the camera via the interface over the network 1504. For example, a regulatory official may wish to view the treatment location before authorizing research and testing or before granting regulatory approval. A determination is made whether the party is permitted access by accessing permissions 1506. At least a portion of this information is persistently stored to compile a record of activities at the treatment location. This record can be useful to prove activities after the fact. The activities that are recorded may drive workflow and scheduling at the treatment location to improve efficiency. If the party is permitted access, access is given to the party so that they may receive a captured image or video data 1508. Otherwise, access to the camera by the party is denied 1510.

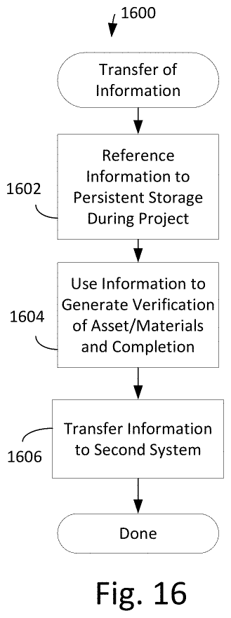

A great deal of information may be collected and stored during a medical activity or associated event for reference during authentication. FIG. 16 shows a flowchart of steps 1600 that may be performed in exemplary embodiments in relation to the information. The information obtained about the article can be derived from many different sources and may be stored on or referenced from immutable storage system 1602. This information may help in the verifications, proof, or other activity concerning authentication and validation of medical devices, tasks, medicines, research, results, and the like. Since there is a complete record including an audit trail on the immutable storage system of all transactions, individuals, activities, tasks, locations, and the like that are associated with the article, these records may be accessed to resolve any issues of authentication, verifications, results, and the like. Insurance providers may access these records referenced on the immutable storage system to provide insurance or confirm claims. Inspection records may be accessed to confirm that proper inspections were carried out and passed. Various records may be accessed to confirm that proper processes, materials, tasks, and the like were carried out and passed so that regulatory approval can be granted. An approval can be made and stored at 1604. The approval can be based upon the article, medication, medical device, task, event, action and its transactions. These items and events can be stored on the immutable storage system.

The record may hold information such as the article, design, materials, transportation, individuals, locations, warranties, confirmation of conditions, Payor information, insurance policy information, inspection history information, ownership history, history of localized events; like environmental conditions and records of trespassing (such as images), and bills of sale as well as receipts for articles. A medical record can include information such as events, transactions, activities, materials, medications, treatments, and the like, between a patient and a healthcare provider. The information in a medical record can include details about the patient's history, clinical findings, diagnostic test results, pre and postoperative care, patient's progress and medication.

The information referenced in the immutable storage system may also be accessed from a computing device of a researcher, distributor, manufacturer, prescriber, user, dispenser, auditor, inspector, auditor, insurance entity, employer, creditor, customer, and the like, at 1606. In exemplary embodiments, information may be gathered from and sent to multiple parties including a company responsible for the management and oversight of a medical device, medicine, or task.

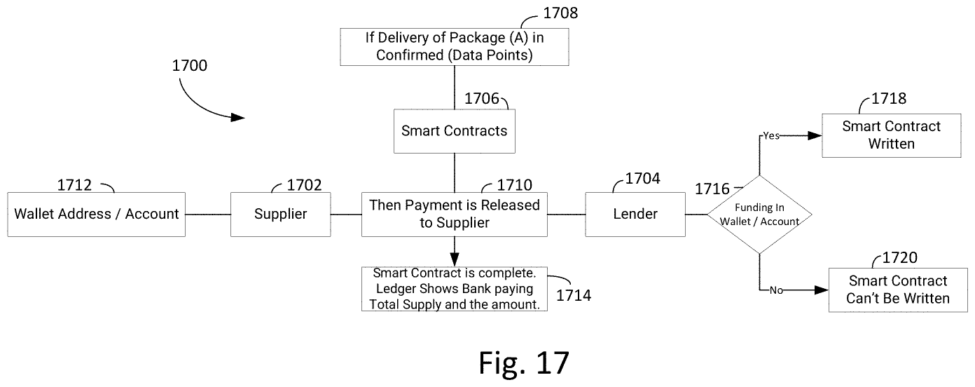

FIG. 17 shows a diagram 1700 of a first example of interactions relating to a smart contract for the medical activity including medical device, medicine, task, process, or project. Smart contacts and automatic payments or reimbursement can be facilitated since the system includes data capture and verifications of medical activities, objects, individuals, and items so that the friction of financial transactions and inefficiencies can be reduced.

By way to example, suppose that the supply company 1702 makes a delivery to the treatment location. Further, suppose that the delivery is confirmed 1708 by information such as that gathered by the system, as discussed above. The payor 1704 then releases payment 1710 to the supply company 1702. Payments can be made through third party funding, factoring, credit lines, loans, or other financial option to assist with financing and cash flow management. Payments can be effectuated with smart contracts when conditions of the smart contact are detected by, retrieved by, inputted into, or otherwise provided to, the system. For example, when a medical provider uses a medical device or performs a treatment, uses medical equipment or distributes medications, the system can record and store this activity and automatically submit a reimbursement request, such as to an insurance company.

The payment may be made electronically, such as through zero trust payment methods including crypto currencies, Bitcoin or Ethereum, fiat currency, electronic account, wallet, or stable coin, whose value is pinned to an item as with fiat currency or the like. Payment may also be in the form of a medical reimbursement. Other suitable forms of electronic payment include Automated Clearing House (ACH) payment, Electronic Funds Transfer (EFT), card payments, other types of bank transfers or other types of electronic wallet transfer. In the case where crypto-currency is used, the crypto-currency may be delivered to the digital wallet of the supply company at a specified wallet address or account 1712. The ledger may be updated to show that the contract is complete 1714. Payment requires that the Payor has sufficient funding in their digital wallet 1716. If not, the smart contract will not be written on the immutable storage system 1718. If there is sufficient funding, payment is made, and the contract is written onto the persistence storage as complete at 1720.

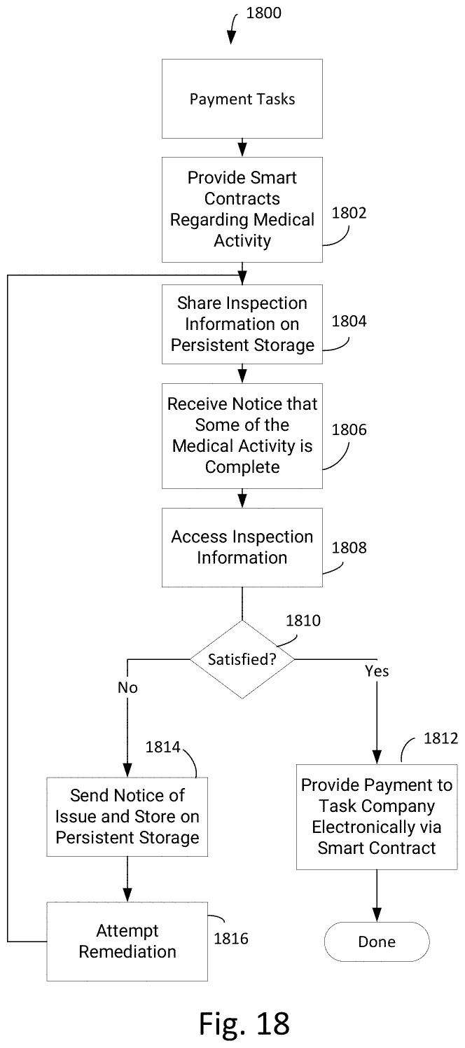

FIG. 18 shows a flowchart 1800 depicting steps performed for tasks of a project or process. Initially a smart contract may be initiated that uses the blockchain-based distributed ledger, where the smart contract is for at least a portion of the medical activity 1802. An inspection, health care provider or auditor can focus on a medical item, event, treatment, or activity. The individual may be required to perform certain tasks of accomplish certain activities under the contract, including a smart contract. The smart contact can be implemented according to the individual actions or data input that can include information representing that the supervisor, auditor or healthcare provider is represented with data that passes through a hash function that can result in a hash value. The hash value may be referenced on the immutable storage system 1804. The information may include, for example, the name of the individual, the date of the inspection, event, activity, and the like, an identification of what occurred, an indication of whether approvals were granted, and the inspection was passed, any notes and an identification of any defects that cause a failed audit, inspection or other review, and how to remedy. A notice is received at the system that a portion of the project is complete 1806. The information is assessed 1808. If the information indicates that the activity was passed 1810 then payment may be provided 1812 to research lab, testing facility or manufacturing facility via smart contract. In contrast, if the event was unsuccessful, a notice of the failure and a notice of issues that need to be addressed may be sent, hashed, and resulting hash value may be referenced on the blockchain-based immutable storage system 1814 for review. The lab, testing facility, treatment facility, provider, or manufacturer may then attempt to remediate the problems 1816 and repeat the above-described steps beginning with new audits, tasks, activities, inspections, and the like, and a reference to a hash value for information regarding the new activity on the immutable storage system 1804.

To pair a medical device or medication with its virtual representation the system captures events at various points from raw materials to final distribution and use. Pairing the physical material with the virtual representation can include several elements or components. Included in the pairing process can be the physical observation of the materials to create a medical device or medication and its components and then associate the medical device or medication with a virtual representation so that the medical device or medication is properly associated with the virtual representation. This verification provides trust that the virtual representation is accurately associated with the medical device or medication as a fact rather than simply trusting that the virtual representation is accurate. This system can use manual or automated processes to physically observe the medical device or medication and its creation tasks and associate the medical device or medication with the virtual representation during various events from raw material to final deliverables. Verification can also use the metadata that is associated with the interaction of medical device or medication by individuals and electronics when the item is created, transported, installed, activated, and destroyed. The metadata that can be captured and placed into immutable storage can provide stakeholders an audit trail of history for their medical device or medication using a verified paired virtual representation. A similar process as described herein can be used for pairing a biometric identifier with an individual.

Verification, including verification of an event, can include verifying that the physical material and the virtual representation match and can be accomplished in a variation of methods including interaction with identification elements such as a tag, label, and the like, capturing an image of the material, capturing a video of the material, capturing a tag physically affixed or otherwise associated with the material, human visual inspection, and any combination. Identification of an individual performing or otherwise associated with an event can be captured by identification devices (e.g., cards, tags, RF ID) and biometrics including visual capture (e.g., facial recognition), voice recognition, iris scan, fingerprint, palm print, and any combination.

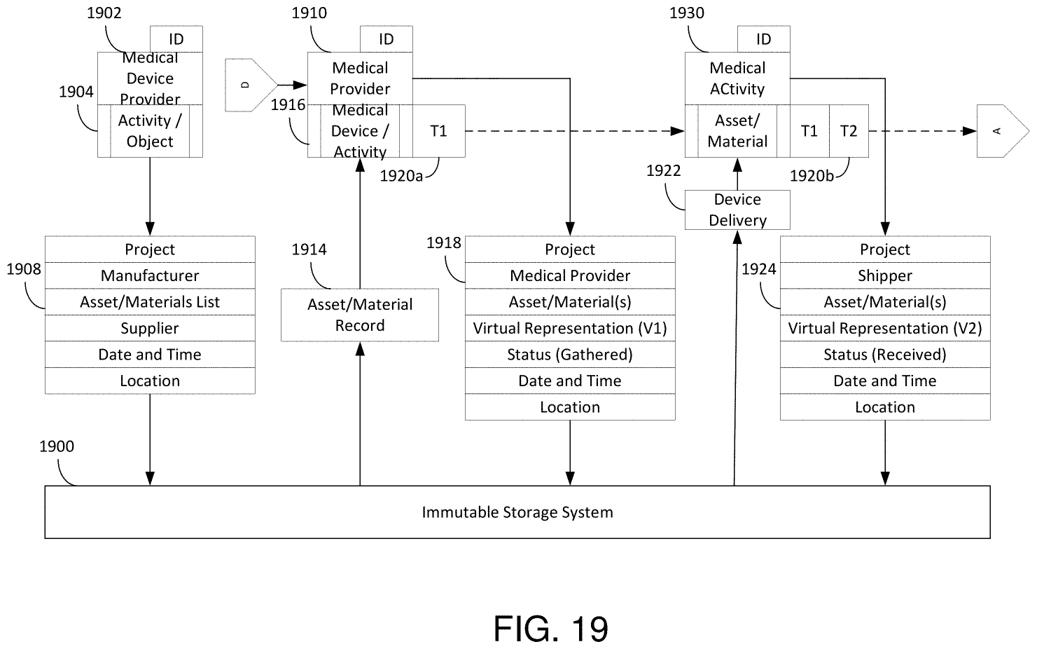

Referring to FIG. 19, an exemplary embodiment is shown. The immutable storage system 1900 is accessible by a medical device provider 1902. The medical provider can have a unique ID associated with the provider. The medical provider can create a medical object record that represents a medical activity. For example, the medical device can be a prosthetic, crutch, implant, and can be for general use or specific use. The medical provider can include a material list and other properties for the medical device or medication. For example, if the medical device is an implant, the medical provider can specify medical grade stainless steel, titanium, and other biomedical materials, tools, and equipment. There are a host of requirements that include multiple American Society for Testing and Materials standards. For example, if the medical device is for a knee replacement, ASTM F1223 provides for the material and test methods for the medical device. The system can create a medical device record 1908 that can include information associated with the material, its manufacturer, is supplier, treatment location, supplier for one or more materials, the date and time the design was created or modified and the location where the design was created or modified and other metadata. The medical device record, manufacturing record, shipping record, and storage record can be stored on the immutable storage system that can be local or remote from the designer.

From this record, a material record can be created and stored on the immutable storage system. The material record can include a single component or multiple components. Each component or material in a set of materials can include a warranty that is from the researcher, tester, manufacturer, distributor, health care professional, installer, and any combination. The requirements for a warranty to remain in place can include requirements such as compliance with installation processes, environmental conditions, use of licensed individuals, use of qualified and experienced individuals, and any combination.

A medical provider, using a computer system 1910, can select or otherwise acquire the medical device 1916 identified on the material list from a materials record 1908 or can be retrieved or otherwise received by the medical manufacturer from the immutable storage system. The medical device supplier can verify that the material matches the material requirement record, and the system can capture this event. For example, one method of associating the physical material with a virtual representation is using a tag 1920a (T₁) placed on the material. The tag is then physically verified to be associated with the material from the material list or the material record. Therefore, the physical material and the virtual representation (V₁) are paired by recording this event and associating the physical material, T₁, and V₁. The material can be received by the manufacturer, scanned, or otherwise identified with a sensor assembly, inspected by an individual and the manufacturing process recorded. This can include capturing the metadata associated with the material, individual, locations, date, time, and process as stated herein. In one embodiment, the tag can include the following information:

| Description | Digits | Information |
|---|---|---|
| Locations | 19-20 | GPS XX.XXXXXX XXX.XXXXXX |
| User ID | 8 | SSN XXXX + Initials XX + Gender X |
| Date | 10 | XX/XX/XXXX |
| Time | 7 | Zulu XXXX:XX |
| Material | 12 | UPC/Barcode XXXXXXXXXXXX |

A medical application record 1918 can be created and stored on the immutable storage system. The capture event can include a unique number and include the supplier ID, date and time, location, material ID, status, and any combination. The material ID can be from an original manufacturer or the supplier. The status can include that the material has been gathered, manufactured, packaged, ordered, is in stock or on back order, shipping information, and any combination. The supplier record can include or reference a designer or manufacturing specification record that can include the material and design requirements. The shipping information can include the origin, destination, shipping instructions, shipper, and any combination thereof and can be included in or reference by the supplier record. The material being the medical device or medication.

The medical provider can apply the medical device or treatment to the patient at the treatment location. When the medical device or activity is used or applied, the computer system can capture this event by creating a medical object 1924 record using a medical provider computer system 1930. The medical provider or other entity can verify the medical activity by methods including adding a tag 1920b (T₂) representing that the proper medical device, medicine, or other object or activity was provided to the proper location. The treatment location computer system can be used to verify that the medical device, medicine, treatment, or other medical activity was properly delivered by retrieving the record 1914 from the immutable storage system and using the record to match the medical activity.

A healthcare provider may have ownership of the medical device or medication and therefore the right to transfer the medical device or medication to a second entity. The transaction can be recorded that can include the device ID of the kiosk, tag information of a tag associate with the medical device or medication, exchangeable image file data or other data associated with a photo of the medical device or medication, biometrics of the health care professional, certifier, auditor, patient, pharmacist, or other individual associated with the medical device or medication, location and time certifications of certifier, wallet confirmation, medical device or medication number, hash confirmation, audit information, location over time for the medical device or medication and other information described herein. The system can also store medical device or medication and transactions associated with those in possession of the medical device or medication and those receiving possession and generate transaction history for the entities, jointly or separately that can include the data described herein.

Using this system, the recipient or other entity can be assured that the medical device or medication was independently verified and authenticated from the creation to the delivery to the patient and that the virtual representation of the medical device or medication and its components (e.g., materials) are paired. Therefore, when purchasing a medical device or medication, the authenticity can be quickly and easily verified at the location of acquisition (e.g., medical facility or pharmacy) by accessing the immutable storage system without the need for a third party or professional authentication process.

The system described herein can pair the medical device, medication, or container with a virtual representation. Failure to pair the medical device, medication, or container with the virtual representation can negatively impact areas such as authentication, certification, fraud prevention, health, legality, possession, abuse, and the like. Tracking, management, and verification of medical device, medication, or container to ensure authenticity and use and manufacturing is an important aspect to many medical device, medication, or containers. Tracking and record keeping during the life of a medical device, medication, or container from its creation to use can be difficult to perform without the ability to pair the medical device, medication, or container with a virtual representation.

Systems at multiple locations may be interconnected using image capture devices, RFID, QR codes, barcodes, biometric scanners, still cameras, video cameras, and the like, to identify individuals or machines that are performing verifications during the process. Further, multiple individuals or machines are performing verifications so that there is not a reliance upon any one entity for verifications. The processing of capturing data, including images, from the multiple systems at multiple locations can be used to improve the verification of proper materials and articles as well as to pair the physical items with the virtual representation.

This process can include internal and external individuals and machines for performing inspections (e.g., verifications). For example, the system can receive a set of internal inspection information entered into the system from an internal inspector representing an internal physical inspection of the medical activity. As an item travels, an internal inspector can provide inspection information representing the stages of the process. The system can also receive a set of external inspection information from an external inspector and an external inspection computer device representing a third-party physical inspection of the process at predetermined stages of the process. Based upon the internal inspection, external inspection or both, an inspection record can be created and stored on the immutable storage system.

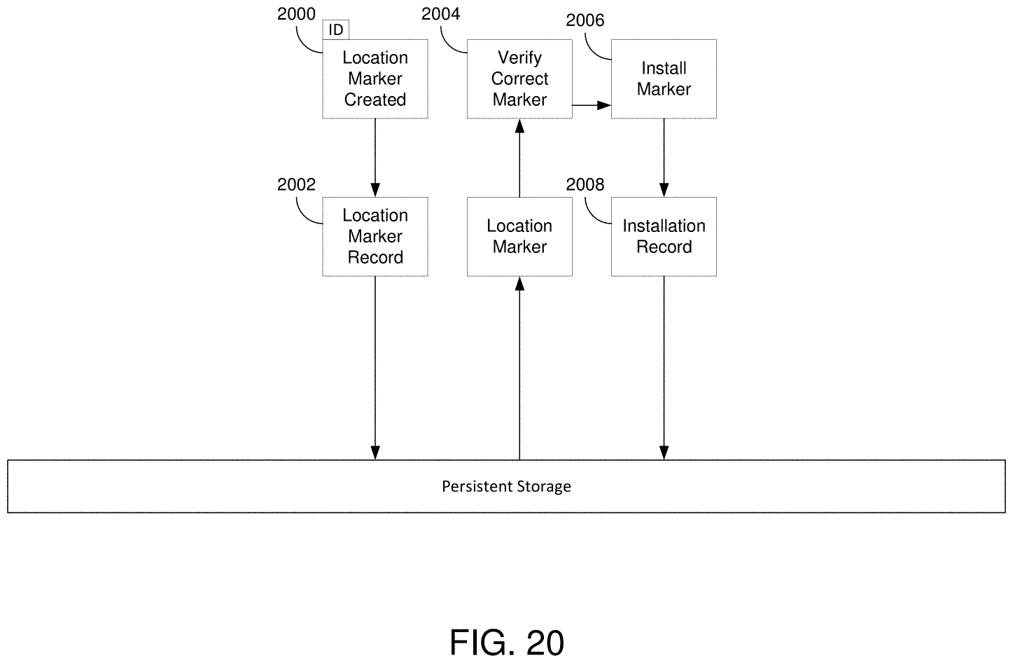

Referring to FIG. 20, a location marker 2000 can be used to uniquely identify a treatment and can include a research facility, testing facility, manufacturing facility, health care facility, pharmacy, retail location, distributor, wholesale location, warehouse, medical device, medication, or container location, other physical location or portion thereof. The location marker can be a barcode, RFID, placard, sign, plaque, QR code, or other symbolic, alphanumeric, digital, geofence, or electronic identifier. When creating the location marker, a location marker record 2002 can be created that includes the location marker identification information, creation date, maker, manufacturing location and other information that can be stored on the immutable storage system. The installed information can retrieve the location marker record and match the retrieved information with the physical location marker to verify that the correct location marker is being installed at 2004. The installed can physically install the location marker and using a location enabled device (e.g., GPS enabled, triangulation, wireless positioning system, and the like), read the location marker, and create a location marker installation record 2008. The installation can be paired with the physical location marker and the physical location of the project. The metadata from the location enabled device can be included in a location marker installation record that can also include installer information, date, time, location marker information and physical location information, and can be stored on the immutable storage system. Therefore, the physical location marker is verified to be paired with the treatment location and a virtual representation of the location market and treatment location.

The system can be contained in one or more computer systems and one or more kiosks that are located at treatment locations such as labs, businesses, manufacturing locations, shipping locations, storage locations, dispensary locations, and the like. The kiosk can be in communication with each other or with the immutable ledger. One or more of the records described herein can be included in an audit trail associated with the medical device or medicine. The audit trail can be localized or disparate. The records that can define an audit trail can be localized or disparate.

Verification of a medical device, medication, treatment, or medical activity with direct information such as capturing the image of the medical activity at a date, time, and location, capturing the individual at a date, time, and location, and any combination. The data captured can be compared with external data such as occupancy information, access control information, shipping records, receipt records, and the like. When the captured data is compared with external data, discrepancies can be detected which can assist in the identification of issues such as fraud, mistakes, and the like. For example, when data about an individual is captured performing a test, the system can correlate the date, time, and location of the test with an access system. If the individual accessed the location where the test occurs, the virtual representation that the individual is at that location can be verified. An image of individuals and article or task can be capture by the system and compared with third-party information such as occupancy, attendance, environment, and other information and if the third-party information is consistent with the transaction information, confidence in the authenticity of the truncation and article increases.

Third parties can use the system, such as an insurance company, certifier, authentication company, and the like. The medical object record can be stored on the immutable storage system so that the data, event, activity, or item information cannot be subsequently tampered with without detection. The record can include a virtual representation of information associated with the medical device, medication, treatment, or other medical item or activity. In one embodiment, the medical activity can be specified by class, type, product code, product number of other identifying information and virtual representation so that the material requirement record includes the article paired with the virtual representation which is verified by the certifier. This verification is independent of the other verifications described herein.

The medical object record can include a medical device manufacturing place, warehouse site, shipping information, transaction information or other information showing the life of an article, device, or task. For example, the medical object record can include the manufacturing location, serial number, part number, product number, series number, lot number, date shipped, and date recited at a location. The article or task record can include when the article was used (e.g., during a medical treatment), the location where the article will be used, the location of the task and what the article or task will ultimately be used for.

When the medical device, medication, or container or component (e.g., article) is selected for transport by the manufacturer or supplier, a shipping company can be sent a shipping order representing the article to be transported. The shipping order can include the verifiably paired virtual representation and its information. The shipping company can be provided shipping information from the manufacturer, designer, supplier, or other company that can facilitate the transaction (e.g., broker, distributor, reseller). The shipping order can be provided directly to the shipping company or can be retrieved from the immutable storage system. Once the shipping company receives the shipping order, it can travel to the desired article location and determine if the article in the shipping order match the medications or container at the desired location. A shipping pickup record can be created representing that the shipping company received the article and that the article picked matches the shipping order. This verification can be independent of the other verifications described herein.

Once that article is delivered to the desired location, the shipping company can unload the article and verify that the article that was unloaded was the same article included in the shipping order. The shipping company can create a shipping delivery record representing that the article was delivered and that the article matches the shipping order. The shipping record can include the environmental conditions when the article was delivered, delivery notes, and the like. The shipping delivery record can be stored on the immutable storage system. Receiving entity can review the article delivered and verify that the delivered article matches the design, article requirements, supply record, shipping order, shipping pickup record, shipping delivery record, or any combination.

Once the article is received by the desired location, the system can notify individuals that the article is ready for distribution. The individuals can be verified by the system and an individual verification record can be created and stored on the immutable storage system. The system can utilize biometrics or other systems as described herein for verification of actual individuals at the physical location to correspond with requirement for those that can verify that the article is authorized.

The system can identify individuals including when individuals are entering or leaving a location and store this information on the immutable storage system. The information capture can identify individuals through biometric identification devices such as a camera or other image capture device, facial recognition, voice recognition, retinal scans, fingerprint scanners, hand scanners, and other biometric devices. In one embodiment, the computing logic may allow authorized individuals to manually enter the presence of another authorized individual, including on the controller at the treatment location or through a remote device that can be determined to be at the treatment location, within a boundary associated with the treatment location, in proximity to the system. In one embodiment, individuals may be verified and paired with a virtual representation using two-factor authentication.

The individual can be provided with user information and specifications or other design requirements that can be represented by a task record. The task record can be stored on the immutable storage system. Once installed, the system can verify that the article was used (which can be individually inspected) according to the task record, create a task verification record and store the task verification record on the immutable storage system. The activity verification record represents that a task associated with the article was performed and can signify that the activity was completed. The activity record can represent that the activity was performed by a proper individual and in compliance with any requirements as well as if the article passes one or more inspections.

Prior to, during and after a task is completed, an inspection can be performed that can include a pre-task inspection, task inspection, post task inspection and any combination. A pre-task inspection, task inspection, and post task inspection record can be created so that the three records can be stored on the immutable storage system. The task record can include information that the inspection resulted in passing, passing with deficiencies, and failing. If the inspection fails, a medical supervisor, or other entity can be given the opportunity to remedy the failure and the inspection process can be performed again. The process can also determine if, while the task passed the inspection, the deficiencies should be remedied.

The system can be uniquely associated with the treatment location. A location marker can be affixed to the user location and uniquely identify the treatment location. The treatment location can be a research facility, lab, warehouse, shipper, distributor, storage area, health care facility, retail location, mobile or temporary medical facility, and the like. The location marker can be read by the system so that the system can determine its location. Third parties can read the location marker to determine the location. For example, the shipping company can arrive at the manufacturing location, read the location marker, and associate the location marker with the receipt and shipping of an article. In one embodiment, the manufacturer can receive a shipping identifier associated with the delivery, such as a truck, trailer, pallet, or other container so that the article is known to be received at the user location. Other parties can also access the location marker to verify that the third party is at the treatment location.

The system can be contained in a housing such as a kiosk and can be physically associated with the treatment location. The treatment location can be defined by a boundary representing the perimeter of the treatment location. The system can include a sensor and reader which can be selected from the group consisting of radio frequency identification (RFID) detector, ultra-high frequency (UHF) detector, a bar code scanner, a QR code scanner, near frequency communication (NFC) device; Bluetooth beacons, an optical character recognition (OCR) device, and any combination thereof. An environmental sensor, such as a weather sensor or weather station, can be in communications with the or included in the housing and configured to record the weather and other environmental conditions at the treatment location and at different times during the project. This information can be used to verify the authenticity of the article.

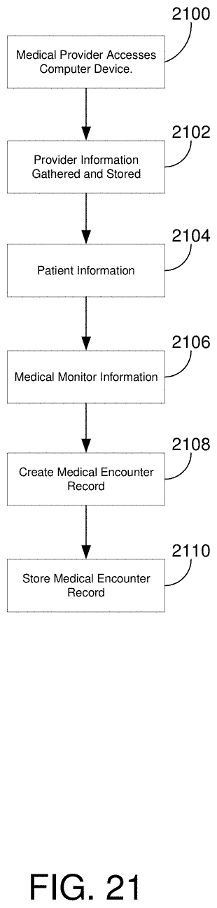

Referring to FIG. 21, a medical provider can use a computer system that can be present at or transported to a treatment location at 2100. The computer system can receive and store medical provider information which can include the date, time, location, and biometric information of the medical provider 2102. The computer system can include a location sensor such as a GPS sensor so that the location of the computer device is known, and the location can be associated with data that is received inputted or otherwise gathered from the computer device. Therefore, the location of the computer device can be known as well as the location of the computer device at a certain date and time.

The computer device can receive medical provider information. This information can be used to determine the identity of the medical provider and location of the medical provider at a certain date and time. This information can be used to create medical provider information that can be recorded on the immutable ledger. This record can be used to verify that the medical provider, who can be identified through biometric information so that verification of the identified is known, was at a certain location at a certain date and time. The medical provider identification can be compared to the computer system information so that a determination can be made the that the medical provider and the computer system were in the same location at a certain date and time. In one embodiment, verification that the computer system and the medical provider were at the same location during a date and time can be determined using a date and time range.

The computer device can receive patient information at 2104. This information can be used to determine the identity of the patient and location of the patient at a certain date and time. This information can be used to create a patient identification information that can be recorded on the immutable ledger. This record can be used to verify that the patient, who can be identified through biometric information so that verification of the identified is known, was at a certain location at a certain date and time. The medical provider identification can be compared to the patient information and computer system information so that a determination can be made the that the medical provider, the patient and the computer system were in the same location at a certain date and time. In one embodiment, verification that the patient and the medical provider were at the same location during a date and time can be determined using a date and time range.

A medical monitor can be in communication with the computer device and can be adapted to receive medical information about the patient at 2106. Information that can be received, measured, inputted, gathered, and the like, can include a vital sign, medical alert, implantable medical information, pumps information, stimulator information, inertial sensor information, physiological information, and any combination thereof. The medical monitor can also record medical treatment, advice, suggestions, application of medical device (e.g., applying a cuff, splint, stent, and any like processes and procedures). The medical monitor and computer system can receive medical information manually such as blood pressure as well as through automated methods such as EEG or others. The computer system and medical monitor can receive input wired, or wirelessly, and can be in communications with such devices as a blood glucose, heart rate, or blood pressure monitor.

The medical activity that can be recorded can include a medical treatment that can be the application of a service such as physical therapy, assessments, testing, diagnostics, surgery, occupational treatment, or other therapy, and the like. Medical activity can be the gathering and recording of medical information such as physiological information. Medical activity can be the provision of medicine or other medical good. Medical activity can be instructions, strategies, or plans that can be delivered to the patient.

From the information gathered, the computer system can create a medical encounter record that can be stored on the immutable storage system. Validations can be made from the medical encounter record that can include verification that the medical provider and the patient were at the same location at the same time and that the medical activity was recorded, reducing the risk that incorrect or even fraudulent information is being submitted such as to the medical computer system, insurance system, and reimbursement systems. The computer system can retrieve information about the medical provider and determine if the medical provider is authorized to provide or participate in the medical activity. In certain circumstances, insurance payments and reimbursement for medical services are contingent that the medical providers have certain licenses and authorization. The computer system can generate a warning when a medical activity is performed by a non-licensed and non-authorized medical provider, at a non-authorized location, within or outside a predetermined treatment time, with non-authorized medical device, medicines or items, and the like. Such warning can be provided to the medical provider so that the medical activity can be assigned to another medical provider. Such warning and controls can assist in the reduction or elimination of unapproved medical facilities, medical activities that are not covered by insurance, non-approved medical devices or treatments, medical activity that is not in compliance with applicable policies, regulations, approvals, and the like, as well as otherwise non-approved medical activities.

When the media encounter record is stored at 2110, the computer system and a remote computer system can retrieve the medical encounter record and analyze the medical encounter record for a determination of validity, reimbursement, regulatory compliance, policy and procedure compliance, and other characteristics. The medical provider can be apprised of any discrepancies in the process by the treatment location thereby reducing the amount of time dedicated to correction of any actual or potential issues.

The medical location can include any location, stationary or mobile, where medical services are provided and can include a testing facility, medical facility, healthcare provider facility, manufacturing facility, distribution facility, pharmacy, residence, assisted living facility, room or location within a facility, inpatient facility or portion thereof, and any combination thereof.

The medical activity can be assigned a unique medical identification. When the medical activity includes providing medicines and a medical device, the medicine and medical device can include a tag that identifies the medicine or medical device. The tag can be uniquely associated with the medicine or the medical device.

This computer system and a remote system can determine if the medical provider, medical activity, and patient were at the same location at the same or near same time. Further, capturing biometric information of the individual reduces the misidentification of the medical provider and patient. Mistakes and even fraud are reduced as the verification represents which individual preformed what tasks at what date and time. For example, the computer system can determine its location when patient information is entered. The computer system can then retrieve patient information from a local or remote database and retrieve the home or other location of the patient. If the computer system location and the patient home location do not match, a warning can be generated. This feature can reduce or prevent medical activities from being performed at unauthorized locations, can indicate that the patient information may not be accurate or that a mistake or fraud is occurring. The combinations of identification (including biometric identification) coupled with location, date and time of the individuals and activities can reduce or eliminate mistakes and fraud as well as provide validation and verification that the digital representations accurately reflect the physical, activities, individuals, and objects. In one embodiment, the computer system can track an individual or object from one location to the next. For example, a medical provider can be at a first location with a first patient and subsequently at a second location with a second patient. The distance and travel time between the first location and the second location can be calculated or approximated and compared with the timespan between when the medical provider was at the first location and second location. A determination can then be made concerning the feasibility of the activities of the medical provider. The ability to prove that a medical activity was performed at the first location and the second location with a sufficient timespan can assist with identifying a mistake or fraud. Further, using biometric information, the system allows for the identity of the provider and the patient to be known.

In one embodiment, the identification device and the medical monitor can determine their own location and can have date and time information which can be compared with the location, date, and time of the computer system to provide a more accurate data set.

In operation, the medical activity can include a medical procedure, administering a medicine, applying a medical device, providing medical instructions, a medical assessment, a medical validation, and any combination thereof. The medical activity can be the application of a splint (e.g., medical device), administering medications (e.g., providing medicine), provide advice or recommendations (e.g., medical advice), and any combination.

When data, including the medical encounter record is stored on the immutable storage system, a remote computer system can retrieve the record and preform an analysis on the data. The computer system and the remote computer system can use the medical encounter record and create a medical reimbursement request that can be used to send to a reimbursement agent as the medical encounter record can be used to verify that the medical activity was performed, who performed the activity, who received the activity, where the medical activity was performed and when it was performed using various points of identification, date, time, and location. This information can increase the time between the medical activity and the reimbursement process.

The computer system and the remote system can use the immutable storage system and retrieve multiple records for the provider, patient, activity, location, and the like. When compared, a medical provider can be shown to perform the medical activity within a short time frame or arrival at the treatment location which be used to create and modify a confident score. The confidence score can be a representation for the medical provider that the information associated with the medical provider is accurate. If a medical provider has larger gaps between arrival and the treatment location and the medical activity, the confidence score can be lower.

The medical provider does not need to be a human. In one embodiment, the medical monitor can be an autonomous medical sensor and artificial intelligence device, such as a telemedicine device, such as within a kiosk. The telemedicine device can include a patient interface, sensors, communications, and storage. When the telemedicine is actuated by the patient, the telemedicine device can capture its locations and the date and time. A human medical provider can be remotely located from the patient and can use the telemedicine device to view medical information and communication with the patient. The human medical provider can access the remote telemedicine device that can capture the identity of the remote medical provider and the date, time, and location of the remote medical provider. The remote medical provider information can be captured and used for storage and verification. For example, the time between the telemedicine sessions can be used to assist in a determination whether a mistake is made or even fraud given that medical activity information can be compared with the remote medical provider information.

Data, including the medical encounter record, can be sent to a medical provider, and can be included in an electronic medical record. The electronic medical record can be provided with a pointer (e.g., a memory location) within an immutable storage system so that the medical information can be stored more securely without multiple copies of the information in multiple databases. The pointer can be removed when access to the immutable storage system is revoked.

The individuals on the treatment location may be prompted to wear certain wearables, including medically sterilized wearables, that provide useful information to the computer system. For instance, individuals may be prompted to wear location tracking devices, such as GPS devices, Bluetooth, radio frequency identification (RFID) devices, ultra-high frequency (UHF) and beacon-based devices. The use of the wearables helps to perform geofencing within the treatment location. The location tracking provided by the wearable helps the system to monitor the location of individuals on the treatment location on an ongoing basis. The permissions may define what portions of the treatment location an individual may access. Ongoing monitoring may indicate that an individual is attempting to enter a location where the individual is not permitted. This may trigger a response as described herein. A signal may be sent to the vest or wearable to trigger a visual or audible cue that the individual is not in a permitted area. In addition, individuals may be requested to wear wearables that track biometric information, such as heart rate, body temperature, respiration rate, and blood pressure. This information may be tracked and stored on an ongoing basis.

The system may track the movement of articles at the treatment location or to and from the treatment location. Scanning technology such as RFID readers, UHF readers, and the like may be utilized to assist the location tracking of articles and even individuals. The tracking of articles helps reduce the risk of loss, theft, mis-delivery, and the like. For example, the tracking solution may indicate instances of possible theft, such as when the article is leaving the treatment location when the removal of the article is not proper. Tracking can improve the confidence in the authentication of the article.

The system may allow for the establishment of one or more geofenced zones that can be associated with treatment location and location of the article. These locations can include entrance areas, exit areas, event areas, storage areas, and any combination thereof. These areas could be monitored and established with access allowances or restrictions to control movement of articles, individuals, and equipment to assist with the prevention of loss, mistakes, fraud, theft, inefficiencies, and damage. The system can assist with verification that articles stored at these locations are consistent with the information concerning the article status, locations, state, etc.

The system, including a controller, may also interface with individuals to allow for the entry of notes and related details of a material, task, inspection, environmental conditions, individual, other task, process of individual, or any combination thereof. For example, the system may allow an inspector to capture images of notes, forms, documents, labels, and the like using various readers, sensors, and input devices. The system can capture the use of the object such as during a medical treatment or application of a medical device.

The system can be adapted to register a medication and drug, medical device, or medical article or object at its point of origin which can include specifications and conformity issues not only for the transportation and handling of the article, but also can confirm with the integrity of manufacturing and transportation, that at the time of dosage or treatment, administration, or use, a set of requirements related to that medical article are met. The system provides for confirmation of delivery to a set physical location (e.g., identifiable location such as with GPS technology). The medical article is then registered to that location. At the time of an event, the system registers the location, date, time, individual, and articles using an immutable block chain or other immutable storage system mechanism, registers the biometrics of the administrator or caregiver, as well as the biometrics of the person receiving the medical article or treatment. The system can immutably store this data associated with an asset record or other unique identifying record. These data points then can be used by the system to autonomously validate processes, payments, or progress points in an overall process. The system can include an auditing capability whereby when these data points are immutably recorded in the asset record, currently by a hash on an immutable block chain, the auditing functionality verifies the hash recorded in the asset record is recorded upon the referenced or corresponding block chain. The auditing component verifies the data recorded at the hash is the same data in the asset record results in a system that can identify anomalies and provide for predictive analysis.

What is claimed is:

1. A computerized system for management of patient encounters comprising:
a medical encounter computer system in communications with an immutable storage system;
a sensor in communication with the medical encounter computer system;
a medical device in communication with the medical encounter computer system adapted to record vital signs of a patient;
a healthcare provider record stored on the immutable storage system;
a patient record stored on the immutable storage system;
a set of computer readable instructions included in the medical encounter computer system adapted to:
receive healthcare provider identifying information from the sensor, retrieve the healthcare provider record from the immutable storage system and generate a healthcare provider notification according to a comparison of the healthcare provider identifying information with the healthcare provider record,
receive patient identifying information from the sensor, retrieve the patient record from the immutable storage system and generate a patient notification according to a comparison of the patient identifying information with the patient record,
receive a healthcare activity information from the sensor and generate a healthcare activity notification according to a comparison of the healthcare activity information with the healthcare provider record, and,
generate a medical device notification according to a comparison of a medical device usage with the healthcare provider record.

2. The system of claim 1 wherein the sensor is adapted to receive biometric information.

3. The system of claim 1 wherein the healthcare provider identifying information includes a healthcare provider location and the set of computer readable instructions are adapted to receive a medical device location from the medical device and generate a medical device location notification according to a comparison of the healthcare provider location with the medical device location.

4. The system of claim 1 wherein the patient identifying information includes a patient location and the set of computer readable instructions are adapted to receive a medical device location from the medical device and generate a medical device location notification according to a comparison of the patient location with the medical device location.

5. The system of claim 1 wherein the patient identifying information includes a patient location, the medical encounter computer system includes a medical encounter computer system location, and the set of computer readable instructions are adapted to generate a medical device location notification according to a comparison of the patient location and the medical encounter computer system location.

6. The system of claim 1 wherein the medical encounter computer system includes a medical encounter computer system location, and the set of computer readable instructions are adapted to generate a medical device location notification according to a comparison of a healthcare provider location received from the medical encounter computer system and the medical encounter computer system location.

7. The system of claim 1 wherein the medical encounter computer system includes a medical encounter computer system location, and the set of computer readable instructions are adapted to generate a medical device location notification according to a comparison of a patient location included in the patient record and the medical encounter computer system location.

8. A computerized system for management of patient encounters comprising:
a medical encounter computer system in communications with an immutable storage system;
a sensor in communication with the medical encounter computer system;
a healthcare provider record stored on the immutable storage system;
a patient record stored on the immutable storage system;
a set of computer readable instructions included in the medical encounter computer system adapted to:
receive healthcare provider identifying information from the sensor, retrieve the healthcare provider record from the immutable storage system and generate a healthcare provider notification according to a comparison of the healthcare provider identifying information and the healthcare provider record,
receive patient identifying information from the sensor, retrieve the patient record from the immutable storage system and generate a patient notification according to a comparison of the patient identifying information and the patient record, and,
receive a healthcare activity information from the sensor and generate a healthcare activity notification according to a comparison of the healthcare activity information and the healthcare provider record.

9. The system of claim 8 wherein the healthcare provider record includes healthcare provider authorization, and the set of computer readable instructions are adapted for generating an authorization notification according to a comparison of the healthcare provider record and the healthcare activity information.

10. The system of claim 8 wherein the set of computer readable instructions are adapted to receive a treatment location from the sensor, receive a healthcare provider location from the sensor, receive a patient location from the sensor and generate a location notification according to a comparison of the healthcare provider location, patient location information and healthcare activity location.

11. The system of claim 8 wherein the set of computer readable instructions are adapted to receive a healthcare provider arrival time, receive a healthcare provider departure time, calculate an on-location time, and generate a temporal notification according to a comparison of the on-location time and the healthcare activity information.

12. The system of claim 11 wherein the set of computer readable instructions are adapted to append the on-location time with the patient record to create an appended patient record and store the appended patient record on the immutable storage system.

13. The system of claim 11 wherein the healthcare provider arrival time is a first healthcare provider arrival time, and the set of computer readable instructions are adapted to:
receive a first location associated with the healthcare provider departure time,
receive a second healthcare provider arrival time at a second location,
calculate a travel time according to the first location and the second location, and
generate a travel notification according to a comparison of the healthcare provider departure time, the second healthcare provider arrival time and the travel time.

14. The system of claim 8 wherein the set of computer readable instructions are adapted to receive a healthcare provider arrival time, receive a healthcare provider current location information, retrieve a healthcare provider prior location information from the immutable storage system, calculate a travel time according to the healthcare provider arrival time, and generate a travel notification according to a comparison of the healthcare provider arrival time and the travel time.

15. The system of claim 14 wherein the healthcare provider prior location information is a prior activity information taken from a group consisting of parking information, transaction information, travel information, event information, mobile device location, mode of travel, and any combination thereof.

16. The system of claim 8 wherein the set of computer readable instructions are adapted to create a medical encounter record according to the healthcare provider identifying information, the patient identifying information and the healthcare activity information.

17. The system of claim 8 wherein the sensor is adapted to retrieve a medical device information from a medical device associated with a patient.

18. The system of claim 17 wherein the set of computer readable instructions are adapted to retrieve a medication record from a medication computer system and generates a medication notification according to a comparison of the medication record and the patient record.

19. A computerized system for management of patient encounters comprising:
a medical encounter computer system in communications with an immutable storage system;
a sensor in communication with the medical encounter computer system;
a healthcare provider record stored on the immutable storage system;
a medication record stored on the immutable storage system;
a set of computer readable instructions included in the medical encounter computer system adapted to:
receive healthcare provider identifying information from the sensor, retrieve the healthcare provider record from the immutable storage system and generates a healthcare provider notification according to a comparison of the healthcare provider identifying information and the healthcare provider record,
retrieve the medication record and generate a medication notification according to a comparison of a patient record with the medication record, and
receive a healthcare activity information from the sensor, retrieves a medical regulatory record from a regulatory computer system and generates a regulatory notification according to a comparison of the healthcare activity information and the medical regulatory record.

20. The system of claim 19 wherein the set of computer readable instructions are adapted to retrieve a healthcare activity time from the medical encounter computer system and generates a medication notification according to a comparison of the healthcare activity time with the medication record.

21. The system of claim 19 wherein the set of computer readable instructions are adapted to retrieve a patient record from the immutable storage system and generates a medication notification according to a comparison of the patient record and the medication record.

22. A computerized system for management of patient encounters comprising:
a medical encounter computer system in communications with immutable storage system;
a sensor in communication with the medical encounter computer system;
a healthcare provider record stored on the immutable storage system;
a set of computer readable instructions included in the medical encounter computer system adapted to:
receive healthcare provider identifying information from the sensor, retrieve the healthcare provider record from the immutable storage system and generate a healthcare provider notification according to a comparison of the healthcare provider identifying information and the healthcare provider record, and,
receive a healthcare activity information from the sensor, retrieve a medical regulatory record from a regulatory computer system and generate a regulatory notification according to a comparison of the healthcare activity information with the medical regulatory record.

23. The system of claim 22 wherein the set of computer readable instructions are adapted to receive a treatment location from the medical encounter computer system and generate a treatment location notification according to a comparison of the treatment location and the medical regulatory record.

24. The system of claim 22 wherein the set of computer readable instructions are adapted to receive a healthcare provider arrival time, receive a healthcare provider departure time, calculate an on-location time, and generate a temporal notification according to a comparison of the on-location time and the healthcare activity information.

25. The system of claim 22 wherein the set of computer readable instructions are adapted to receive a healthcare provider arrival time, receive a healthcare provider departure time, calculate an on-location time, and generate a temporal notification according to a comparison of the on-location time and the medical regulatory record.

26. The system of claim 22 wherein the healthcare activity information is a first healthcare activity information included in a set of healthcare activity information stored on the immutable storage system and the set of computer readable instructions are adapted for creating a token representing a portion of the set of healthcare activity information and providing the token to a third party.

27. The system of claim 22 wherein the set of computer readable instructions are adapted for effectuating a medical transaction according to the healthcare activity information.

28. The system of claim 27 wherein the medical transaction is an effectuation of a smart contract.

29. The system of claim 27 wherein the medical transaction is placed on a queue for approval prior to its effectuation.

30. The system of claim 22 wherein the set of computer readable instructions are adapted to retrieve warranty information for a medical device used in the medical activity and generates a warranty notification according to a comparison of the warranty information and the healthcare provider identifying information.
